(19) United States
(12) Patent Application Publication
(10) Pub. No.: US 2004/0235100 A1
(43) Pub. Date: Nov. 25, 2004

(54) Renilla reniformis green fluorescent protein and mutants thereof

(75) Inventors: Joseph A. Sorge, Wilson, WY (US); Peter E. Vaillancourt, Del Mar, CA (US); Katherine A. Felts, San Diego, CA (US)

Correspondence Address: PALMER & DODGE, LLP, KATHLEEN M. WILLIAMS / STR, 111 HUNTINGTON AVENUE, BOSTON, MA, 02199, US

(73) Assignee: Stratagene

(21) Appl. No.: 10/786,425

(22) Filed: Feb. 25, 2004

Related U.S. Application Data
(62) Division of application No. 09/795,040, filed on Feb. 26, 2001.
(60) Provisional application No. 60/210,561, filed on Jun. 9, 2000.
(60) Provisional application No. 60/185,589, filed on Feb. 28, 2000.

(51) Int. Cl. C12Q 1/68; C07H 21/04; C12N 5/08

(52) U.S. Cl. 435/69.1; 435/320.1; 435/325; 530/350; 435/366; 536/23.2; 435/252.3

(57) ABSTRACT

The invention relates to recombinant polynucleotides encoding the Green Fluorescent Protein (GFP) from R. reniformis, as well as polynucleotides encoding variants and fusion polypeptides of R. reniformis GFP. The invention further relates to vectors encoding R. Reniformis GFP and variants and fusions thereof, as well as to cells comprising and/or expressing such vectors. The invention also relates to recombinant R. reniformis GFP polypeptides and fusion polypeptides and variants thereof, as well as to methods of making and using such polypeptides both in vivo and in vitro.

This application is a divisional of U.S. Ser. No. 09/795,040, filed Feb. 26, 2001, which claims priority to U.S. Provisional applications No. 60/210,561, files Jun. 9, 2000 and 60/185,589, filed Feb. 28, 2000. The contents of these applications are incorporated herein by reference.

### BACKGROUND OF THE INVENTION

The green fluorescent protein (GFP) from the jellyfish Aequorea victoria has become an extremely useful tool for tracking and quantifying biological entities in the fields of biochemistry, molecular and cell biology, and medical diagnostics (Chalfie et al., 1994, Science 263: 802-805; Tsien, 1998, Ann, Rev. Biochem. 67: 509-544). There are no cofactors or substrates required for fluorescence, thus the protein can be used in a wide variety of organisms and cell types. GFP has been used as a reporter gene to study gene expression in vivo by insertion downstream of a test promoter. The protein has also been used to study the subcellular localization of a number of proteins by direct fusion of the test protein to GFP, and GFP has become the reporter of choice for monitoring the infection efficiency of viral vectors both in cell culture and in animals. In addition, a number of genetic modifications have been made to GFP resulting in variants for which spectral shifts correspond to changes in the cellular environment such as pH, ion flux, and the phosphorylation state of the cell. Perhaps the most promising role for GFP as a cellular indicator is its application to fluorescence resonance energy transfer (FRET) technology. FRET occurs with fluorophores for which the emission spectrum of one overlaps with the excitation spectrum of the second. When the fluorophores are brought into close proximity, excitation of the “donor” fluorophore results in emission from the “acceptor”. Pairs of such fluorophores are thus useful for monitoring molecular interactions. Fluorescent proteins such as GFP or variants thereof are useful for analysis of protein:protein interactions in vivo or in vitro if their fluorescent emission and excitation spectra overlap to allow FRET. The donor and acceptor fluorescent proteins may be produced as fusions with the proteins one wishes to analyze for interactions. These types of applications of GFPs are particularly appealing for high throughput analyses, since the readout is direct and independent of subcellular localization.

Purified A. victoria GFP is a monomeric protein of about 27 kDa that absorbs blue light with excitation wavelength maximum of 395 nm, with a minor peak at 470 nm, and emits green fluorescence with an emission wavelength of about 510 nm and a minor peak near 540 nm (Ward et al., 1979, Photochem. Photobiol. Rev. 4: 1-57). The excitation maximum of A. victoria GFP is not within the range of wavelengths of standard fluorescein detection optics. Further, the breadth of the excitation and emission spectra of the A. victoria GFP are not well suited for use in applications involving FRET. In order to be useful in FRET applications, the excitation and emission spectra of the fluorophores are preferably tall and narrow, rather than low and broad. There is a need in the art for GFP proteins that are amenable to the use of standard fluorescein excitation and detection optics. There is also a need in the art for GFP proteins with narrow, preferably non-overlapping spectral peaks.

The use of A. victoria GFP as a reporter for gene expression studies, while very popular, is hindered by relatively low quantum yield (the brightness of a fluorophore is determined as the product of the extinction coefficient and the fluorescence quantum yield). Generally, the A. victoria GFP coding sequences must be linked to a strong promoter, such as the CMV promoter or strong exogenous regulators such as the tetracycline transactivator system, in order to produce readily detectable signal. This makes it difficult to use GFP as a reporter for examining the activity of native promoters responsive to endogenous regulators. Higher intensity would obviously also increase the sensitivity of other applications of GFP technology. There is a need in the art for GFP proteins with higher quantum yield.

Another disadvantage of A. victoria GFP involves fluctuations in its spectral characteristics with changes in pH. At high pH (pH 11-12), the wild-type A. victoria GFP loses absorbance and excitation amplitude at 395 nm and gains amplitude at 470 nm (Ward et al., 1982, Photochem. Photobiol. 35: 803-808). A. victoria fluorescence is also quenched at acid pH, with a pKa around 4.5. There is a need in the art for GFPs exhibiting fluorescence that is less sensitive to pH fluctuations.

Further, in order to be more useful in a broad range of applications, there is a need in the art for GFP proteins exhibiting increased stability of fluorescence characteristics relative to A. victoria GFP, with regard to organic solvents, detergents and proteases often used in biological studies. There is also a need in the art for GFP proteins that are more likely to be soluble in a wider range of cell types and less likely to interfere non-specifically with endogenous proteins than A. victoria GFP.

A number of modifications to A. victoria GFP have been made with the aim of enhancing the usefulness of the protein. For example, modifications aimed at enhancing the brightness of the fluorescence emissions or the spectral characteristics of either the excitation or emission spectra or both have been made. It is noted that the stated aim of several of these modification approaches was to make an A. victoria GFP that is more similar to R. reniformis GFP in its excitation and emission spectra and fluorescence intensity.

Literature references relating to A. victoria mutants exhibiting altered fluorescence characteristics include, for example, the following. Heim et al. (1995, Nature 373: 663-664) relates to mutations at S65 of A. victoria that enhance fluorescence intensity of the polypeptide. The S65T mutation to the A. victoria GFP is said to “ameliorate its main problems and bring its spectra much closer to that of Renilla”.

A review by Chalfie (1995, Photochem. Photobiol. 62: 651-656) notes that an S65T mutant of A. victoria, the most intensely fluorescent mutant of A. victoria known at the time, is not as intense as the R. reniformis GFP.

Further references relating to A. victoria mutants include, for example, Ehrig et al., 1995, FEBS Lett. 367: 163-166); Surpin et al., 1987, Photochem. Photobiol. 45 (Suppl): 95S; Delagrave et al., 1995, BioTechnology 13: 151-154; and Yang et al., 1996, Gene 173: 19-23.

Patent and patent application references relating to A. victoria GFP and mutants thereof include the following. U.S. Pat. No. 5,874,304 discloses A. victoria GFP mutants said to alter spectral characteristics and fluorescence intensity of the polypeptide. U.S. Pat. No. 5,968,738 discloses A. victoria GFP mutants said to have altered spectral characteristics. One mutation, V163A, is said to result in increased fluorescence intensity. U.S. Pat. No. 5,804,387 discloses A. victoria mutants said to have increased fluorescence intensity, particularly in response to excitation with 488 nm laser light. U.S. Pat. No. 5,625,048 discloses A. victoria mutants said to have altered spectral characteristics as well as several mutants said to have increased fluorescence intensity. Related U.S. Pat. No. 5,777,079 discloses further combinations of mutations said to provide A. victoria GFP polypeptides with increased fluorescence intensity. International Patent Application (PCT) No. WO98/21355 discloses A. victoria GFP mutants said to have increased fluorescence intensity, as do WO97/20078, WO97/42320 and WO97/11094. PCT Application No. WO98/06737 discloses mutants said to have altered spectral characteristics, several of which are said to have increased fluorescence intensity.

In addition to A. victoria, GFPs have been identified in a variety of other coelenterates and anthazoa, however only two GFPs have been cloned, those from A. victoria (Prasher, 1992, Gene 111: 229-233) and from the sea pansy, Renilla mulleri (WO 99/49019).

### SUMMARY OF THE INVENTION

The invention encompasses recombinant polynucleotides encoding the GFP from R. reniformis, as well as polynucleotides encoding variants and fusion polypeptides of R. reniformis GFP, as well as methods of using such polynucleotides and polypeptides.

More particularly, the invention encompasses a recombinant polynucleotide which comprises the sequence of SEQ ID NO: 1.

In one embodiment, the recombinant polynucleotide which comprises the sequence of SEQ ID NO: 1 further comprises a sequence encoding at least one fused heterologous polypeptide domain.

The invention further encompasses a recombinant vector comprising a polynucleotide sequence encoding R. reniformis GFP.

In one embodiment, the sequence encoding R. reniformis GFP is SEQ ID NO: 1.

In another embodiment the recombinant vector is selected from the group consisting of a plasmid, a bacteriophage, a virus, and a retrovirus.

The invention further encompasses a cell comprising a recombinant polynucleotide encoding R. reniformis GFP.

The invention further encompasses a cell comprising a recombinant vector comprising a polynucleotide sequence encoding R. reniformis GFP, or the polynucleotide sequence of SEQ ID NO: 1.

The invention further encompasses an isolated recombinant polypeptide comprising the amino acid sequence of SEQ ID NO: 2.

The invention further encompasses a recombinant polypeptide comprising the amino acid sequence of R. reniformis GFP or a variant thereof and at least one fused heterologous polypeptide domain.

In one embodiment, the at least one fused heterologous polypeptide domain is fused to the amino-terminal end of the R. reniformis GFP or variant thereof.

In another embodiment, the at least one fused heterologous polypeptide domain is fused to the carboxy-terminal end of the R. reniformis GFP or variant thereof.

In another embodiment, the at least one fused heterologous polypeptide domain is fused to the R. reniformis GFP or variant thereof via a linker sequence.

The invention further encompasses a method of producing R. reniformis GFP comprising the steps of: a) introducing a recombinant vector comprising a polynucleotide sequence encoding R. reniformis GFP to a cell; b) culturing the cell of step (a); and c) isolating R. reniformis GFP from the cell.

In one embodiment, the cell is a bacterial cell.

In another embodiment, the cell is a eukaryotic cell.

In a preferred embodiment, the eukaryotic cell is selected from the group consisting of yeasts, insect cells, and mammalian cells. It is preferred that the mammalian cells are human.

In another embodiment, the polynucleotide sequence is a humanized sequence.

The invention further encompasses a polynucleotide encoding an altered R. reniformis GFP polypeptide with increased fluorescence intensity relative to wild-type R. reniformis GFP.

In one embodiment, the polypeptide has at least one mutation relative to wild type R. reniformis GFP in the stretch of amino acids defined by amino acids 64-69 of SEQ ID NO: 2.

The invention further encompasses a polynucleotide encoding an R. reniformis GFP polypeptide with an excitation spectrum that is detectably distinct from that of wild-type R. reniformis GFP.

The invention further encompasses a polynucleotide encoding an R. reniformis GFP polypeptide with an emission spectrum that is detectably distinct from that of wild-type R. reniformis GFP.

The invention further encompasses a method of detecting protein:protein interactions, the method comprising the following steps: a) providing a first fusion polypeptide comprising a first polypeptide domain and a first R. reniformis GFP-derived polypeptide, and a second fusion polypeptide comprising a second polypeptide domain and a second R. reniformis GFP-derived polypeptide, wherein the emission spectrum of the first R. reniformis GFP-derived polypeptide overlaps the excitation spectrum of the second R. reniformis GFP-derived polypeptide, the second R. reniformis GFP-derived polypeptide emits fluorescence with a spectrum that is distinguishable from fluorescence emitted by the first R. Reniformis GFP-derived polypeptide, and wherein the first R. reniformis GFP-derived polypeptide may be excited by a spectrum of light that does not excite fluorescence emission by the second R. reniformis GFP-derived polypeptide; b) mixing the first and the second fusion polypeptides; c) irradiating the mixture of step (b) with a spectrum of light that excites the first R. reniformis GFP-derived polypeptide to emit fluorescence but does not excite the second R. reniformis GFP-derived polypeptide; and d) detecting fluorescence emission from the second R. reniformis GFP-derived polypeptide, wherein the fluorescence emission from the second R. reniformis GFP polypeptide indicates protein:protein interaction between the first and the second polypeptide domains.

In one embodiment, the method is performed in a living cell.

The invention further encompasses a method of determining the location of a polypeptide of interest in a cell, wherein a polynucleotide sequence encoding the polypeptide of interest is known, the method comprising the steps of: a) linking the polynucleotide sequence encoding the polypeptide of interest with a polynucleotide encoding R. reniformis GFP, such that the linked polynucleotide sequences are fused in frame; b) introducing the linked polynucleotide sequences to a cell; and c) determining the location of the polypeptide encoded by the linked polynucleotide sequences.

In one embodiment the method is performed in a living cell.

The invention further encompasses a method of identifying cells to which a recombinant vector has been introduced, the method comprising the steps of: a) introducing a recombinant vector to a population of cells, wherein the recombinant vector encodes R. reniformis GFP; b) illuminating the population with light within the excitation spectrum of R. reniformis GFP; and c) detecting fluorescence in the emission spectrum of R. reniformis GFP in the population, thereby identifying a cell to which the recombinant vector has been introduced.

In one embodiment, the GFP is expressed as a fusion polypeptide.

In another embodiment, the GFP is expressed as a distinct polypeptide.

In another embodiment, the cell is identified by FACS analysis.

The invention further encompasses a method of monitoring the activity of a transcriptional regulatory sequence, the method comprising the steps of: a) operably linking a nucleic acid sequence comprising the transcriptional regulatory sequence to a nucleic acid sequence encoding R. reniformis GFP of SEQ ID NO: 2 to form a reporter construct; b) introducing the reporter construct to a cell; and c) detecting R. reniformis GFP fluorescence in the cell, wherein the fluorescence reflects the activity of the transcriptional regulatory sequence.

The invention further encompasses a method of detecting a modulator of a transcriptional regulatory sequence, the method comprising the steps of: a) operably linking a nucleic acid sequence comprising the transcriptional regulatory sequence to a nucleic acid sequence encoding R. reniformis GFP of SEQ ID NO: 2 to form a reporter construct, wherein the transcriptional regulatory sequence is responsive to the presence of the modulator; b) introducing the reporter construct to a cell; and c) detecting R. reniformis GFP fluorescence in the cell, wherein the fluorescence indicates the presence of the modulator.

In one embodiment, the modulator is selected from the group consisting of a hormone or lipid soluble transcriptional modulator, a growth factor, and a heavy metal.

The invention further encompasses a method of screening for an inhibitor of a transcriptional regulatory sequence, the method comprising the steps of: a) operably linking a nucleic acid sequence comprising the transcriptional regulatory sequence to a nucleic acid sequence encoding R. reniformis GFP of SEQ ID NO: 2 to form a reporter construct; b) introducing the reporter construct to a cell; c) contacting the cell with a candidate inhibitor of the transcriptional regulatory sequence; and d) detecting R. reniformis GFP fluorescence in the cell, wherein a decrease in the fluorescence relative to that detected in the absence of the candidate inhibitor indicates that the candidate inhibitor inhibits the activity of the transcriptional regulatory sequence.

The invention further encompasses a method of producing a fluorescent molecular weight marker, the method comprising the steps of: a) linking a nucleic acid sequence encoding R. Reniformis GFP in frame to a nucleic acid sequence encoding a polypeptide of known relative molecular weight such that the linked molecules encode a fusion polypeptide; b) introducing the linked nucleic acid sequences of (a) to a cell; c) isolating the fusion polypeptide from the cell, wherein the fusion polypeptide is a molecular weight marker.

The invention further encompasses a polynucleotide encoding R. reniformis GFP or a variant of R. reniformis GFP, wherein the polynucleotide comprises at least one humanized codon sequence.

The invention further encompasses a humanized polynucleotide, the polynucleotide encoding R. reniformis GFP or a variant of R. reniformis GFP.

In one embodiment, the humanized polynucleotide comprises the sequence of SEQ ID NO: 3.

The invention further encompasses a recombinant vector comprising a humanized R. reniformis GFP polynucleotide.

The invention further encompasses a cell containing a recombinant vector comprising a humanized R. reniformis GFP polynucleotide.

As used herein, the term “R. reniformis green fluorescent protein” or “R. reniformis GFP” refers to a polypeptide of SEQ ID NO: 2 or to a fluorescent variant thereof. An R. reniformis GFP variant encompasses polypeptides of SEQ ID NO: 2 that bear one or more mutations, including insertion or deletion of one or more amino acids, either at the N or C termini of the polypeptide or internal to the coding sequence. Variants of R. reniformis GFP retain the ability to emit light when excited by light within a given part of the spectrum, and may be excited by light of, or emit light in a portion of the spectrum that differs detectably from that which excites or which is emitted by wild-type R. reniformis GFP of SEQ ID NO: 2. In addition to variants exhibiting different excitation or emission spectra, R. reniformis GFP variants include variants exhibiting increased fluorescence intensity relative to wild-type R. reniformis GFP.

The term “variant thereof” when used in reference to an R. reniformis polynucleotide coding sequence means that the sequence bears one or more nucleotide differences relative to the sequence of the wild-type R. reniformis coding sequence. A variant of an R. reniformis polynucleotide sequence encodes an R. reniformis GFP polypeptide or a variant thereof. A variant of an R. reniformis polynucleotide coding sequence includes a humanized polynucleotide coding sequence. A variant polynucleotide directs the expression of an amount of fluorescent polypeptide at least equal to, or greater than, the amount expressed from an equal mass amount or from an equal number of copies of a non-humanized R. reniformis GFP polynucleotide sequence.

The term “humanized polynucleotide” or “humanized sequence” refers to a polynucleotide coding sequence in which one or more, including 5 or more, 10 or more, 20 or more, 50 or more, 75 or more, 100 or more, 125 or more, 150 or more, 200 or more, or even all codons of the polynucleotide coding sequence for a non-human polypeptide (i.e., a polypeptide not naturally expressed in humans) have been altered to a codon sequence more preferred for expression in human cells. Because there are 64 possible combinations of the 4 DNA nucleotides in codon groups of 3, the genetic code is redundant for many of the 20 amino acids. Each of the different codons for a given amino acid encodes the incorporation of that amino acid into a polypeptide. However, within a given species there tends to be a preference for certain of the redundant codons to encode a given amino acid. The “codon preference” of R. reniformis is different from that of humans (this codon preference is usually based upon differences in the level of expression of the tRNAs containing the corresponding anticodon sequences). In order to obtain high expression of a non-human gene product in human cells, it is advantageous to change one or more non-preferred codons to a codon sequence that is preferred in human cells. Table 1 shows the preferred codons for human gene expression. A codon sequence is preferred for human expression if it occurs to the left of a given codon sequence in the table. Optimally, but not necessarily, less preferred codons in a non-human polynucleotide coding sequence are humanized by altering them to the codon most preferred for that amino acid in human gene expression. The amount of fluorescent polypeptide expressed in a human cell from a humanized GFP polynucleotide sequence is at least two-fold greater, on either a mass or a fluorescence intensity scale per cell, than the amount expressed from an equal amount or number of copies of a non-humanized GFP polynucleotide.

As used herein, the term “humanized codon” means a codon sequence, within a polynucleotide sequence encoding a non-human polypeptide, that has been changed to a codon sequence that is more preferred for expression in human cells relative to that codon encoded by the non-human organism from which the non-human polypeptide is derived. Species-specific codon preferences stem in part from differences in the expression of tRNA molecules with the appropriate anticodon sequence. That is, one factor in the species-specific codon preference is the realtionship between a codon and the amount of corresponding anticodon tRNA expressed.

It should be understood that any of the recombinant vectors of the invention may comprise a humanized polynucleotide encoding R. reniformis GFP or a variant thereof. Similarly, any of the cells of the invention may comprise vectors comprising a humanized polynucleotide encoding R. reniformis GFP or a variant thereof. It should also be understood that all claimed methods using polynucleotides encoding R. reniformis GFP may be performed with humanized polynucleotides encoding R. reniformis GFP or variants of R. reniformis GFP. Finally, any R. reniformis GFP polypeptide of the invention may be expressed from a humanized R. reniformis GFP polynucleotide coding sequence.

As used herein, the term “wild-type R. reniformis GFP” refers to a polypeptide of SEQ ID NO: 2.

As used herein, the term “increased fluorescence intensity” or “increased brightness” refers to fluorescence intensity or brightness that is greater than that exhibited by wild-type R. reniformis GFP under a given set of conditions. Generally, an increase in fluorescence intensity or brightness means that fluorescence of a variant is at least 5% or more, and preferably 10%, 20%, 50%, 75%, 100% or more, up to even 5 times, 10 times, 20 times, 50 times or 100 times or more intense or bright than wild-type R. reniformis GFP under a given set of conditions.

As used herein, the term “fused heterologous polypeptide domain” refers to an amino acid sequence of two or more amino acids fused in frame to R. reniformis GFP or a variant thereof. A fused heterologous domain may be linked to the N or C terminus of the R. reniformis GFP polypeptide or variant thereof.

As used herein, the term “fused to the amino-terminal end” refers to the linkage of a polypeptide sequence to the amino terminus of another polypeptide. The linkage may be direct or may be mediated by a short (e.g., about 2-20 amino acids) linker peptide.

As used herein, the term “fused to the carboxy-terminal end” refers to the linkage of a polypeptide sequence to the carboxyl terminus of another polypeptide. The linkage may be direct or may be mediated by a linker peptide.

As used herein, the term “linker sequence” refers to a short (e.g., about 1-20 amino acids) sequence of amino acids that is not part of the sequence of either of two polypeptides being joined. A linker sequence is attached on its amino-terminal end to one polypeptide or polypeptide domain and on its carboxyl-terminal end to another polypeptide or polypeptide domain.

As used herein, the term “excitation spectrum” refers to the wavelength or wavelengths of light that, when absorbed by a fluorescent polypeptide molecule of the invention, causes fluorescent emission by that molecule.

As used herein, the term “emission spectrum” refers to the wavelength or wavelengths of light emitted by a fluorescent polypeptide.

As used herein, the terms “distinguishable” or “detectably distinct” mean that standard filter sets allow either the excitation of one form of a polypeptide without excitation of another given polypeptide, or similarly, that standard filter sets allow the distinction of the emission from one polypeptide form from the emission spectrum of another. Generally, distinguishable or detectably distinct excitation or emission spectra have peaks that vary by more than 1 nm, and preferably vary by more than 2, 3, 4, 5, 10 or more nm.

As used herein, the term “fusion polypeptide” refers to a polypeptide that is comprised of two or more amino acid sequences, from two or more proteins that are not found linked in nature, that are physically linked by a peptide bond.

As used herein, the term “emission spectrum overlaps the excitation spectrum” means that light emitted by one fluorescent polypeptide is of a wavelength or wavelengths that causes excitation and emission by another fluorescent polypeptide.

As used herein, the term “population of cells” refers to a plurality of cells, preferably, but not necessarily of same type or strain.

As used herein the term “distinct polypeptide” refers to a polypeptide that is not expressed as a fusion polypeptide.

As used herein, the term “FACS analysis” refers to the method of sorting cells, fluorescence activated cell sorting, wherein cells are stained with or express one or more fluorescent markers. In this method, cells are passed through an apparatus that excites and detects fluorescence from the marker(s). Upon detection of fluorescence in a given portion of the spectrum by a cell, the FACS apparatus allows the separation of that cell from those not expressing that fluorescence spectrum.

As used herein, the term “lipid soluble transcriptional modulator” refers to a composition that is capable of passing through cell membranes (nuclear or cytoplasmic) and has a positive or negative effect on the transcription of one or more genes or constructs.

As used herein, the term “operably linked” means that a given coding sequence is joined to a given transcriptional regulatory sequence such that transcription of the coding sequence occurs and is regulated by the regulatory sequence.

As used herein, the term “reporter construct” refers to a polynucleotide construct encoding a detectable molecule, linked to a transcriptional regulatory sequence conferring regulated transcription upon the polynucleotide encoding the detectable molecule. A detectable molecule is preferably an R. reniformis GFP or variant thereof.

As used herein, the term “responsive to the presence of a modulator” means that a given transcriptional regulatory sequence is either turned on or turned off in the presence of a given compound. As used herein, gene expression is “turned on” when the polypeptide encoded by the gene sequence (e.g., a GFP polypeptide or variant thereof) is detectable over background, or alternatively, when the polypeptide is detectable in an increased amount over the amount detected in the absence of a given modulator compound. In this context, “increased amount” means at least 10%, preferably 20%, 50%, 75%, 100% or more, up to even 5 times, 10 times, 20 times, 50 times, or 100 times or more higher than background detection, with background detection being the amount of signal observed in the absence of the modulator compound.

As used herein, the term “modulator of a transcriptional regulatory sequence” refers to a compound or chemical moiety that causes a change in the level of expression from a transcriptional regulatory sequence. Preferably, the change is detectable as an increase or decrease in the detection of a reporter molecule or reporter molecule activity, with at least 10%, 20%, 50%, 75%, 100%, or even 5 times, 10 times, 20 times, 50 times or 100 times or more increased or decreased level of reporter signal relative to the absence of a given modulator.

As used herein the term “inhibitor of a transcriptional regulatory sequence” refers to a compound or chemical moiety that causes a decrease in the amount of a reporter molecule or reporter molecule activity expressed from a given transcriptional regulatory sequence. As used herein, the term “decrease” when used in reference to the detection of a reporter molecule or reporter molecule activity means that detectable activity is reduced by at least 10%, 20%, 50%, 75%, or even 100% (i.e., no expression), relative to the amount detected in the absence of a given compound or chemical moiety. As used herein the term “candidate inhibitor” refers to a compound or chemical moiety being tested for inhibitory activity in an assay.

### BRIEF DESCRIPTION OF THE DRAWINGS

FIG. 1 shows the coding sequence of R. reniformis GFP, SEQ ID NO: 1.

FIG. 2 shows the amino acid sequence of R. reniformis GFP, SEQ ID NO: 2.

FIG. 3 is a graphical representation of R. reniformis GFP expressed in transduced cells. The unshaded peak represents the uninfected cell population; the shaded peak represents cells transduced with the GFP-expressing virus. In this experiment, 44% of the transduced population showed fluorescence above background.

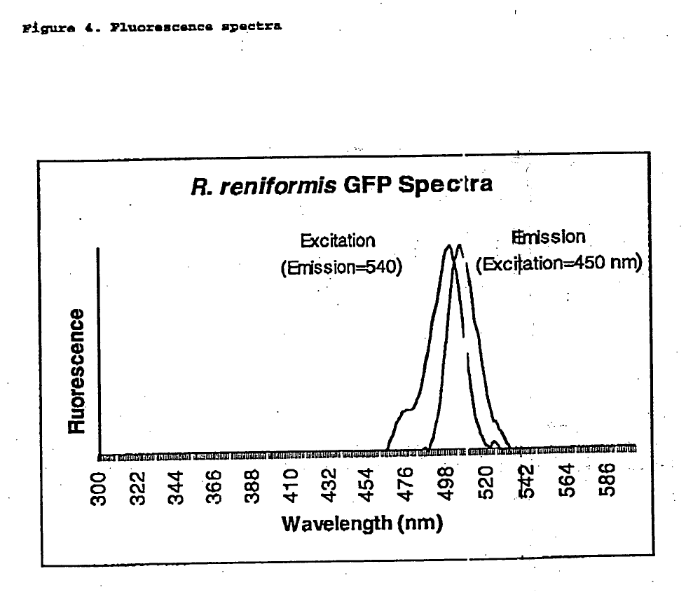

FIG. 4 shows fluorescence spectra of recombinant R. reniformis GFP. Spectra were measured using 10 mn bandwidths. The y-axis scales for the two peaks have been normalized so that the fluorescence profiles have equal amplitude.

FIG. 5 shows the sequence of a humanized R. reniformis GFP polynucleotide sequence (SEQ ID NO: 3).

FIG. 6 shows a sequence alignment between non-humanized and humanized R. reniformis GFP. Vertical lines represent homology between the humanized and non-humanized genes. Gaps represent nucleotides that were altered to produce the hrGFP gene.

FIG. 7 shows the relative fluorescence of CHO cells transduced by retroviral vectors harboring non-humanized or humanized R. reniformis GFP. Cells were infected with undiluted supernatants containing virus derived from the two GFP vectors, or media alone (No Virus).

FIG. 8 shows the relative fluorescence of 293 cells harboring single copy proviral integrants from which either rGFP, hrGFP or EGFP is expressed. The % UR value indicates the number of cells which fluoresce above background. The raw % UR for the “No Virus” control was 0.15%, and was subtracted from the values for all cell populations.

### DESCRIPTION

The invention relates to the GFP from R. reniformis. Polynucleotide sequences encoding the R. reniformis GFP are disclosed herein, as are polypeptide sequences for R. reniformis GFP and variants thereof.

R. reniformis GFP polynucleotides were isolated through PCR amplification using an R. reniformis cDNA library prepared in lambda phage. Full length coding sequences were isolated, sequenced, and inserted into a variety of different expression vectors.

Also disclosed herein are methods of producing R. reniformis GFP polypeptides or variants thereof, the methods comprising introducing an expression vector encoding R. reniformis GFP or a variant thereof into a cell, culturing the cell, and isolating the GFP polypeptides or variants.

I. How to Make R. reniformis GFP Polynucleotides and Polypeptides according to the Invention

A number of methodologies were combined to provide the invention disclosed herein, including molecular, cellular and biochemical approaches. Polynucleotides encoding R. reniformis GFP are obtained in any of several different ways, including direct chemical synthesis, library screening and PCR amplification. R. reniformis GFP polypeptides are obtained by expression from recombinant polynucleotide sequences in appropriate organisms. Useful variants of R. reniformis GFP polypeptides are produced in similar ways following the introduction of mutations to the polynucleotide sequence encoding wild-type R. reniformis GFP. Those methodologies necessary to make and use the R. reniformis GFP polynucleotides, polypeptides and variants thereof of the invention are discussed in detail below.

A. Isolation of R. reniformis GFP-encoding Polynucleotide Sequences

1. R. reniformis cDNA Library Preparation

Construction methods for libraries in a variety of different vectors, including, for example, bacteriophage, plasmids, and viruses capable of infecting eukaryotic cells are well known in the art. Any known library production method resulting in largely full-length clones of expressed genes may be used to provide a template for the isolation of GFP-encoding polynucleotides from R. reniformis.

For the library used to isolate the GFP-encoding polynucleotides disclosed herein, the following method was used. Poly(A) RNA was prepared from R. reniformis organisms as described by Chomczynski, P. and Sacchi, N. (1987, Anal. Biochem. 162: 156-159). cDNA was prepared using the ZAP-cDNA Synthesis Kit (Stratagene cat.# 200400) according to the manufacturer's recommended protocols and inserted between the EcoR I and Xho I sites in the vector Lambda ZAP II. The resulting library contained 5×10⁶ individual primary clones, with an insert size range of 0.5-3.0 kb and an average insert size of 1.2 kb. The library was amplified once prior to use as template for PCR reactions.

2. Isolation of R. reniformis GFP Coding Sequence by PCR

The R. reniformis GFP coding sequence was isolated by polymerase chain reaction (PCR) amplification of the sequence from within the cDNA library described herein. A large number of PCR methods are known to those skilled in the art. Thermal-cycled PCR (Mullis and Faloona, 1987, Methods Enzymol., 155: 335-350; see also, PCR Protocols, 1990, Academic Press, San Diego, Calif., USA for a review of PCR methods) uses multiple cycles of DNA replication catalyzed by a thermostable, DNA-dependent DNA polymerase to amplify the target sequence of interest. Briefly, oligonucleotide primers are selected such that they anneal on either side and on opposite strands of a sequence to be amplified. The primers are annealed and extended using a template-dependent thermostable DNA polymerase, followed by thermal denaturation and annealing of primers to both the original template sequence and the newly-extended template sequences, after which primer extension is performed. Repeating such cycles results in exponential amplification of the sequences between the two primers.

In addition to thermal cycled PCR, there are a number of other nucleic acid sequence amplification methods that may be used to amplify and isolate a GFP-encoding polypeptide according to the invention from an R. reniformis cDNA library. These include, for example, isothermal 3SR (Gingeras et al., 1990, Annales de Biologie Clinique, 48(7): 498-501; Guatelli et al., 1990, Proc. Natl. Acad. Sci. U.S.A., 87: 1874), and the DNA ligase amplification reaction (LAR), which permits the exponential increase of specific short sequences through the activities of any one of several bacterial DNA ligases (Wu and Wallace, 1989, Genomics, 4: 560). The contents of both of these references are incorporated herein in their entirety by reference.

To amplify a sequence encoding R. reniformis GFP from an R. reniformis cDNA library, the following approach was taken. The R. reniformis GFP coding sequence was amplified using the 5′ primer 5′-AATTATTAGAATTCACCATGGTGAGTAAACAAATATTGAAGAAC-3′ and the 3′ primer 5′-ATAATATTCTCGAGTTAAACCCATTCGTGTAAGGATCC-3. The 5′ primer contains an EcoR I recognition site to facilitate subsequent cloning of the amplified fragment, followed by the Kozak consensus translation initiation sequence ACCATGG. The 3′ primer contains an Xho I recognition site to facilitate cloning of the amplified fragment. Oligonucleotides may be purchased from any of a number of commercial suppliers (for example, Life Technologies, Inc., Operon Technologies, etc.). Alternatively, oligonucleotide primers may be synthesized using methods well known in the art, including, for example, the phosphotriester (see Narang, S. A., et al., 1979, Meth. Enzymol., 68:90; and U.S. Pat. No. 4,356,270), phosphodiester (Brown, et al., 1979, Meth. Enzymol., 68:109), and phosphoramidite (Beaucage, 1993, Meth. Mol. Biol., 20:33) approaches. Each of these references is incorporated herein in its entirety by reference.

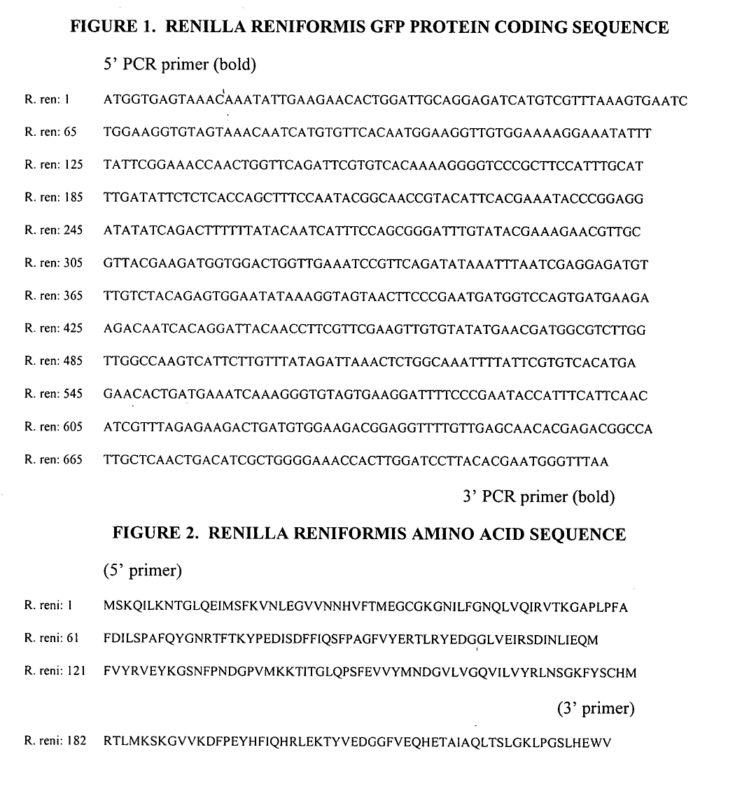

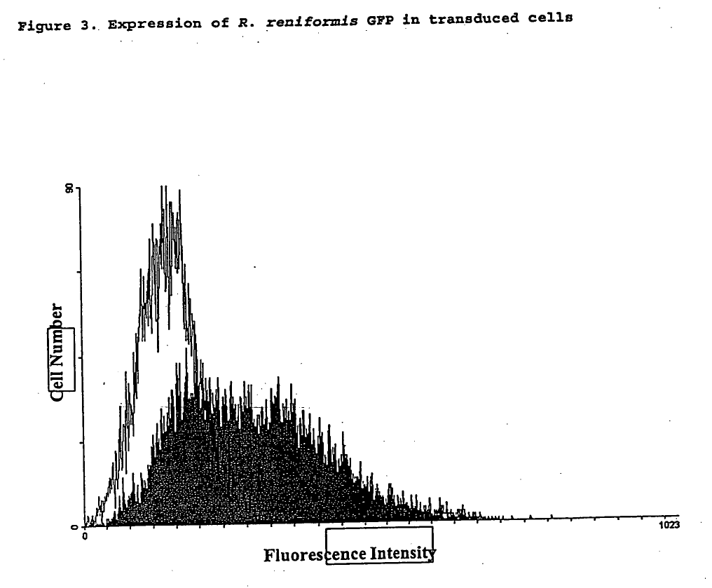

PCR was carried out in a 50 μl reaction volume containing 1× TaqPlus Precision buffer (Stratagene), 250 μM of each dNTP, 200 nM of each PCR primer, 2.5 U TaqPlus Precision enzyme (Stratagene) and approximately 3×10⁷ lambda phage particles from the amplified cDNA library described above. Reactions were carried out in a Robocycler Gradient 40 (Stratagene) as follows: 1 min at 95° C. (1 cycle), 1 min at 95° C., 1 min at 53° C., 1 min at 72° C. (40 cycles), and 1 min at 72° C. (1 cycle). Reaction products were resolved on a 1% agarose gel, and a band of approximately 700 bp was excised and purified using the StrataPrep DNA Gel Extraction Kit (Stratagene). Other methods of isolating and purifying amplified nucleic acid fragments are well known to those skilled in the art. The PCR fragment was subcloned by digestion to completion with EcoRI and XhoI and insertion into the retroviral expression vector pFB (Stratagene) to create the vector pFB-rGFP. Both strands of the cloned GFP fragment were completely sequenced. The coding polynucleotide and amino acid sequences are presented in FIGS. 1 and 2, respectively. The R. reniformis and R. mulleri GFP coding sequences are 83% homologous, and the proteins share 88% identical amino acid sequence.

3. Isolation of R. reniformis GFP-encoding Polynucleotides by Library Screening

An alternative method of isolating GFP-encoding polynucleotides according to the invention involves the screening of an expression library, such as a lambda phage expression library, for clones exhibiting fluorescence within the emission spectrum of GFP when illuminated with light within the excitation spectrum of GFP. In this way clones may be directly identified from within a large pool. Standard methods for plating lambda phage expression libraries and inducing expression of polypeptides encoded by the inserts are well established in the art. Screening by fluorescence excitation and emission is carried out as described herein below using either a spectrofluorometer or even visual identification of fluorescing plaques. With either method, fluorescent plaques are picked and used to re-infect fresh cultures one or more times to provide pure cultures, from which GFP insert sequences may be determined and sub-cloned.

As another alternative, if a sequence is available for the polynucleotide one wishes to obtain, the polynucleotide may be chemically synthesized by one of skill in the art. The same synthetic methods used for the preparation of oligonucleotide primers (described above) may be used to synthesize gene coding sequences for GFPs of the invention. Generally this would be performed by synthesizing several shorter sequences (about 100 nt or less), followed by annealing and ligation to produce the full length coding sequence.

B. Production of R. reniformis GFP Polypeptides and Variants Thereof

The production of R. reniformis GFP polypeptides (e.g., the polypeptide with the amino acid sequence of SEQ ID NO: 2) and variants thereof from recombinant vectors comprising GFP-encoding polynucleotides of the invention may be effected in a number of ways known to those skilled in the art. For example, plasmids, bacteriophage or viruses may be introduced to prokaryotic or eukaryotic cells by any of a number of ways known to those skilled in the art. Following introduction of R. reniformis GFP-encoding polynucleotides to a prokaryotic or eukaryotic cell, expressed GFP polypeptides may be isolated using methods known in the art or described herein below. Useful vectors, cells, methods of introducing vectors to cells and methods of detecting and isolating GFP polypeptides and variants thereof are also described herein below.

1. Vectors Useful according to the Invention

There is a wide array of vectors known and available in the art that are useful for the expression of GFP polypeptides or variants thereof according to the invention. The selection of a particular vector clearly depends upon the intended use of the GFP polypeptide or variant thereof. For example, the selected vector must be capable of driving expression of the polypeptide in the desired cell type, whether that cell type be prokaryotic or eukaryotic. Many vectors comprise sequences allowing both prokaryotic vector replication and eukaryotic expression of operably linked gene sequences.

Vectors useful according to the invention may be autonomously replicating, that is, the vector, for example, a plasmid, exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors.

Vectors useful according to the invention preferably comprise sequences operably linked to the GFP coding sequences that permit the transcription and translation of the GFP sequence. Sequences that permit the transcription of the linked GFP sequence include a promoter and optionally also include an enhancer element or elements permitting the strong expression of the linked sequences. The term “transcriptional regulatory sequences” refers to the combination of a promoter and any additional sequences conferring desired expression characteristics (e.g., high level expression, inducible expression, tissue- or cell-type-specific expression) on an operably linked nucleic acid sequence.

The selected promoter may be any DNA sequence that exhibits transcriptional activity in the selected host cell, and may be derived from a gene normally expressed in the host cell or from a gene normally expressed in other cells or organisms. Examples of promoters include, but are not limited to the following: A) prokaryotic promoters—E. coli lac, tac, or trp promoters, lambda phage P_{R} or P_{L} promoters, bacteriophage T7, T3, Sp6 promoters, B. subtilis alkaline protease promoter, and the B. stearothermophilus maltogenic amylase promoter, etc.; B) eukaryotic promoters—yeast promoters, such as GAL1, GAL4 and other glycolytic gene promoters (see for example, Hitzeman et al., 1980, J. Biol. Chem. 255: 12073-12080; Alber & Kawasaki, 1982, J. Mol. Appl. Gen. 1: 419-434), LEU2 promoter (Martinez-Garcia et al., 1989, Mol Gen Genet. 217: 464-470), alcohol dehydrogenase gene promoters (Young et al., 1982, in Genetic Engineering of Microorganisms for Chemicals, Hollaender et al., eds., Plenum Press, NY), or the TPI1 promoter (U.S. Pat. No. 4,599,311); insect promoters, such as the polyhedrin promoter (U.S. Pat. No. 4,745,051; Vasuvedan et al., 1992, FEBS Lett. 311: 7-11), the P10 promoter (Vlak et al., 1988, J. Gen. Virol. 69: 765-776), the Autographa californica polyhedrosis virus basic protein promoter (EP 397485), the baculovirus immediate-early gene promoter gene 1 promoter (U.S. Pat. Nos. 5,155,037 and 5,162,222), the baculovirus 39K delayed-early gene promoter (also U.S. Pat. Nos. 5,155,037 and 5,162,222) and the OpMNPV immediate early promoter 2; mammalian promoters—the SV40 promoter (Subramani et al., 1981, Mol. Cell. Biol. 1: 854-864), metallothionein promoter (MT-1; Palmiter et al., 1983, Science 222: 809-814), adenovirus 2 major late promoter (Yu et al.,1984, Nucl. Acids Res. 12: 9309-21), cytomegalovirus (CMV) or other viral promoter (Tong et al., 1998, Anticancer Res. 18: 719-725), or even the endogenous promoter of a gene of interest in a particular cell type.

A selected promoter may also be linked to sequences rendering it inducible or tissue-specific. For example, the addition of a tissue-specific enhancer element upstream of a selected promoter may render the promoter more active in a given tissue or cell type. Alternatively, or in addition, inducible expression may be achieved by linking the promoter to any of a number of sequence elements permitting induction by, for example, thermal changes (temperature sensitive), chemical treatment (for example, metal ion- or IPTG-inducible), or the addition of an antibiotic inducing agent (for example, tetracycline).

Regulatable expression is achieved using, for example, expression systems that are drug inducible (e.g., tetracycline, rapamycin or hormone-inducible). Drug-regulatable promoters that are particularly well suited for use in mammalian cells include the tetracycline regulatable promoters, and glucocorticoid steroid-, sex hormone steroid-, ecdysone-, lipopolysaccharide (LPS)- and isopropylthiogalactoside (IPTG)-regulatable promoters. A regulatable expression system for use in mammalian cells should ideally, but not necessarily, involve a transcriptional regulator that binds (or fails to bind) nonmammalian DNA motifs in response to a regulatory agent, and a regulatory sequence that is responsive only to this transcriptional regulator.

One inducible expression system that is well suited for the regulated expression of a GFP polypeptide of the invention or variant thereof, is the tetracycline-regulatable expression system, which is founded on the efficiency of the tetracycline resistance operon of E. coli. The binding constant between tetracycline and the tet repressor is high while the toxicity of tetracycline for mammalian cells is low, thereby allowing for regulation of the system by tetracycline concentrations in eukaryotic cell culture or within a mammal that do not affect cellular growth rates or morphology. Binding of the tet repressor to the operator occurs with high specificity.

Versions of the tet-regulatable system exist that allow either positive or negative regulation of gene expression by tetracycline. In the absence of tetracycline or a tetracycline analog, the wild-type bacterial tet repressor protein causes negative regulation of genes driven by promoters containing repressor binding elements from the tet operator sequences. Gossen & Bujard (1995, Science 268: 1766-1769; also International patent application No. WO 96/01313) describe a tet-regulatable expression system that exploits this positive regulation by tetracycline. In this system, tetracycline binds to a tet repressor fusion protein, rtTA, and prevents it from binding to the tet operator DNA sequence, thus allowing transcription and expression of the linked gene only in the presence of the drug.

This positive tetracycline-regulatable system provides one means of stringent temporal regulation of the GFP polypeptide of the invention or variant thereof (Gossen & Bujard, 1995, supra). The tet operator (tet O) sequence is now well known to those skilled in the art. For a review, the reader is referred to Hillen & Wissmann (1989) in Protein-Nucleic Acid Interaction, “Topics in Molecular and Structural Biology”, eds. Saenger & Heinemann, (Macmillan, London), Vol. 10, pp 143-162. Typically the nucleic acid sequence encoding the GFP polypeptide is placed downstream of a plurality of tet O sequences: generally 5 to 10 such tet O sequences are used, in direct repeats.

In addition to the tetracycline-regulatable systems, a number of other options exist for the regulated or inducible expression of a GFP polypeptide or variant thereof according to the invention. For example, the E. coli lac promoter is responsive to lac repressor (lacI) DNA binding at the lac operator sequence. The elements of the operator system are functional in heterologous contexts, and the inhibition of lacI binding to the lac operator by IPTG is widely used to provide inducible expression in both prokaryotic, and more recently, eukaryotic cell systems. In addition, the rapamycin-controlled transcriptional activator system described by Rivera et al. (1996, Nature Med. 2: 1028-1032) provides transcriptional activation dependent on rapamycin. That system has low baseline expression and a high induction ratio.

Another option for regulated or inducible expression of a GFP polypeptide or variant thereof involves the use of a heat-responsive promoter. Activation is induced by incubation of cells, transfected with a GFP construct regulated by a temperature-sensitive transactivator, at the permissive temperature prior to administration. For example, transcription regulated by a co-transfected, temperature sensitive transcription factor active only at 37° C. may be used if cells are first grown at, for example, 32° C., and then switched to 37° C. to induce expression.

Tissue-specific promoters may also be used to advantage in GFP-encoding constructs of the invention. A wide variety of tissue-specific promoters is known. As used herein, the term “tissue-specific” means that a given promoter is transcriptionally active (i.e., directs the expression of linked sequences sufficient to permit detection of the polypeptide product of the promoter) in less than all cells or tissues of an organism. A tissue specific promoter is preferably active in only one cell type, but may, for example, be active in a particular class or lineage of cell types (e.g., hematopoietic cells). A tissue specific promoter useful according to the invention comprises those sequences necessary and sufficient for the expression of an operably linked nucleic acid sequence in a manner or pattern that is essentially the same as the manner or pattern of expression of the gene linked to that promoter in nature. The following is a non-exclusive list of tissue specific promoters and literature references containing the necessary sequences to achieve expression characteristic of those promoters in their respective tissues; the entire content of each of these literature references is incorporated herein by reference. Examples of tissue specific promoters useful with the R. Reniformis GFP of the invention are as follows: Bowman et al., 1995 Proc. Natl. Acad. Sci. USA 92,12115-12119 describe a brain-specific transferrin promoter; the synapsin I promoter is neuron specific (Schoch et al., 1996 J. Biol. Chem. 271, 3317-3323); the necdin promoter is post-mitotic neuron specific (Uetsuki et al., 1996 J. Biol. Chem. 271, 918-924); the neurofilament light promoter is neuron specific (Charron et al., 1995 J. Biol. Chem. 270, 30604-30610); the acetylcholine receptor promoter is neuron specific (Wood et al., 1995 J. Biol. Chem. 270, 30933-30940); the potassium channel promoter is high-frequency firing neuron specific (Gan et al., 1996 J. Biol. Chem 271, 5859-5865); the chromogranin A promoter is neuroendocrine cell specific (Wu et al., 1995 A.J. Clin. Invest. 96, 568-578); the Von Willebrand factor promoter is brain endothelium specific (Aird et al., 1995 Proc. Natl. Acad. Sci. USA 92, 4567-4571); the flt-1 promoter is endothelium specific (Morishita et al., 1995 J. Biol. Chem. 270, 27948-27953); the preproendothelin-1 promoter is endothelium, epithelium and muscle specific (Harats et al., 1995 J. Clin. Invest. 95, 1335-1344); the GLUT4 promoter is skeletal muscle specific (Olson and Pessin, 1995 J. Biol. Chem. 270, 23491-23495); the Slow/fast troponins promoter is slow/fast twitch myofibre specific (Corin et al., 1995 Proc. Natl. Acad. Sci. USA 92, 6185-6189); the -Actin promoter is smooth muscle specific (Shimizu et al., 1995 J. Biol. Chem. 270, 7631-7643); the Myosin heavy chain promoter is smooth muscle specific (Kallmeier et al., 1995 J. Biol. Chem. 270, 30949-30957); the E-cadherin promoter is epithelium specific (Hennig et al., 1996 J. Biol. Chem. 271, 595-602); the cytokeratins promoter is keratinocyte specific (Alexander et al., 1995 B. Hum. Mol. Genet. 4, 993-999); the transglutaminase 3 promoter is keratinocyte specific (J. Lee et al., 1996 J. Biol. Chem. 271, 4561-4568); the bullous pemphigoid antigen promoter is basal keratinocyte specific (Tamai et al., 1995 J. Biol. Chem. 270, 7609-7614); the keratin 6 promoter is proliferating epidermis specific (Ramirez et al., 1995 Proc. Natl. Acad. Sci. USA 92, 4783-4787); the collagen 1 promoter is hepatic stellate cell and skin/tendon fibroblast specific (Houglum et al., 1995 J. Clin. Invest. 96, 2269-2276); the type X collagen promoter is hypertrophic chondrocyte specific (Long & Linsenmayer, 1995 Hum. Gene Ther. 6, 419-428); the Factor VII promoter is liver specific (Greenberg et al., 1995 Proc. Natl. Acad. Sci. USA 92, 12347-1235); the fatty acid synthase promoter is liver and adipose tissue specific (Soncini et al., 1995 J. Biol. Chem. 270, 30339-3034); the carbamoyl phosphate synthetase I promoter is portal vein hepatocyte and small intestine specific (Christoffels et al., 1995 J. Biol. Chem. 270, 24932-24940); the Na—K—Cl transporter promoter is kidney (loop of Henle) specific (Igarashi et al., 1996 J. Biol. Chem. 271, 9666-9674); the scavenger receptor A promoter is macrophages and foam cell specific (Horvai et al., 1995 Proc. Natl. Acad. Sci. USA 92, 5391-5395); the glycoprotein IIb promoter is megakaryocyte and platelet specific (Block & Poncz, 1995 Stem Cells 13, 135-145); the yc chain promoter is hematopoietic cell specific (Markiewicz et al., 1996 J. Biol. Chem. 271, 14849-14855); and the CD11b promoter is mature myeloid cell specific (Dziennis et al., 1995 Blood 85, 319-329).

Any tissue specific transcriptional regulatory sequence known in the art may be used to advantage with a vector encoding R. reniformis GFP or a variant thereof.

In addition to promoter/enhancer elements, vectors useful according to the invention may further comprise a suitable terminator. Such terminators include, for example, the human growth hormone terminator (Palmiter et al., 1983, supra), or, for yeast or fungal hosts, the TPI1 (Alber & Kawasaki, 1982, supra) or ADH3 terminator (McKnight et al., 1985, EMBO J. 4: 2093-2099).

Vectors useful according to the invention may also comprise polyadenylation sequences (e.g., the SV40 or Ad5E1b poly(A) sequence), and translational enhancer sequences (e.g., those from Adenovirus VA RNAs). Further, a vector useful according to the invention may encode a signal sequence directing the recombinant polypeptide to a particular cellular compartment or, alternatively, may encode a signal directing secretion of the recombinant polypeptide.

Coordinate expression of different genes from the same promoter in a recombinant vector maybe achieved by using an IRES element, such as the internal ribosomal entry site of Poliovirus type 1 from pSBC-1 (Dirks et al., 1993, Gene 128:247-9). Internal ribosome binding site (IRES) elements are used to create multigenic or polycistronic messages. IRES elements are able to bypass the ribosome scanning mechanism of 5′ methylated Cap-dependent translation and begin translation at internal sites (Pelletier and Sonenberg, 1988, Nature 334: 320-325). IRES elements from two members of the picanovirus family (polio and encephalomyocarditis) have been described (Pelletier and Sonenberg, 1988, supra), as well an IRES from a mammalian message (Macejak and Sarnow, 1991 Nature 353: 90-94). Any of the foregoing may be used in an R. reniformis GFP vector in accordance with the present invention.

IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. In this manner, multiple genes, one of which will be an R. reniformis GFP gene, can be efficiently expressed using a single promoter/enhancer to transcribe a single message. Any heterologous open reading frame can be linked to IRES elements. In the present context, this means any selected protein that one desires to express and any second reporter gene (or selectable marker gene). In this way, the expression of multiple proteins could be achieved, for example, with concurrent monitoring through GFP production.

A vector useful according to the invention may also comprise a selectable marker allowing identification of a cell that has received a functional copy of the GFP-encoding gene construct. In its simplest form, the GFP sequence itself, linked to a chosen promoter may be considered a selectable marker, in that illumination of cells or cell lysates with the proper wavelength of light and measurement of emitted fluorescence at the expected wavelength allows detection of cells that express the GFP construct. In other forms, the selectable marker may comprise an antibiotic resistance gene, such as the neomycin, bleomycin, zeocin or phleomycin resistance genes, or it may comprise a gene whose product complements a defect in a host cell, such as the gene encoding dihydrofolate reductase (DHFR), or, for example, in yeast, the Leu2 gene. Alternatively, the selectable marker may, in some cases be a luciferase gene or a chromogenic substrate-converting enzyme gene such as the β-galactosidase gene.

GFP-encoding sequences according to the invention may be expressed either as free-standing polypeptides or frequently as fusions with other polypeptides. It is assumed that one of skill in the art can, given the polynucleotide sequences disclosed herein (e.g., SEQ ID NO: 1) readily construct a gene comprising a sequence encoding R. reniformis GFP or a fluorescent variant thereof and a sequence comprising one or more polypeptides or polypeptide domains of interest. It is understood that the fusion of GFP coding sequences and sequences encoding a polypeptide of interest maintains the reading frame of all polypeptide sequences involved. As used herein, the term “polypeptide of interest” or “domain of interest” refers to any polypeptide or polypeptide domain one wishes to fuse to a GFP molecule of the invention. The fusion of a GFP polypeptide of the invention with a polypeptide of interest may be through linkage of the GFP sequence to either the N or C terminus of the fusion partner, or the GFP sequence may even be inserted in frame between the N and C termini of the polypeptide of interest, if so desired. Fusions comprising GFP polypeptides of the invention need not comprise only a singel polypeptide or domain in addition ot the GFP. Rather, any number of domains of interest may be linked in any way as long as the GFP coding region retains its reading frame and the encoded polypeptide retains fluorescence activity under at least one set of conditions. One non-limiting example of such conditions includes physiological salt concentration (i.e., aboutr 90 mM), pH near neutral and 37° C.

a. Plasmid Vectors

Any plasmid vector that allows expression of a GFP coding sequence of the invention in a selected host cell type is acceptable for use according to the invention. A plasmid vector useful in the invention may have any or all of the above-noted characteristics of vectors useful according to the invention. Plasmid vectors useful according to the invention include, but are not limited to the following examples: Bacterial—pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, and pRIT5 (Pharmacia); Eukaryotic—pWLneo, pSV2cat, pOG44, pXT1, pSG (Stratagene) pSVK3, pBPV, pMSG, and pSVL (Pharmacia). However, any other plasmid or vector may be used as long as it is replicable and viable in the host.

b. Bacteriophage Vectors

There are a number of well known bacteriophage-derived vectors useful according to the invention. Foremost among these are the lambda-based vectors, such as Lambda Zap II or Lambda-Zap Express vectors (Stratagene) that allow inducible expression of the polypeptide encoded by the insert. Others include filamentous bacteriophage such as the M13-based family of vectors.

c. Viral Vectors

A number of different viral vectors are useful according to the invention, and any viral vector that permits the introduction and expression of sequences encoding R. reniformis GFP or variants thereof in cells is acceptable for use in the methods of the invention. Viral vectors that can be used to deliver foreign nucleic acid into cells include but are not limited to retroviral vectors, adenoviral vectors, adeno-associated viral vectors, herpesviral vectors, and Semliki forest viral (alphaviral) vectors. Defective retroviruses are well characterized for use in gene transfer (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals. Details of retrovirus production and host cell transduction of use in the methods of the invention are also presented in Example 1, below.

In addition to retroviral vectors, Adenovirus can be manipulated such that it encodes and expresses a gene product of interest but is inactivated in terms of its ability to replicate in a normal lytic viral life cycle (see for example Berkner et al., 1988, BioTechniques 6:616; Rosenfeld et al., 1991, Science 252:431-434; and Rosenfeld et al., 1992, Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Adeno-associated virus (AAV) is a naturally occurring defective virus that requires another virus, such as an adenovirus or a herpes virus, as a helper virus for efficient replication and a productive life cycle. (For a review see Muzyczka et al., 1992, Curr. Topics in Micro. and Immunol. 158:97-129). An AAV vector such as that described in Traschin et al. (1985, Mol. Cell. Biol. 5:3251-3260) can be used to introduce nucleic acid into cells. A variety of nucleic acids have been introduced into different cell types using AAV vectors (see, for example, Hermonat et al., 1984, Proc. Natl. Acad. Sci. USA 81: 6466-6470; and Traschin et al., 1985, Mol. Cell. Biol. 4: 2072-2081).

Finally, the introduction and expression of foreign genes is often desired in insect cells because high level expression may be obtained, the culture conditions are simple relative to mammalian cell culture, and the post-translational modifications made by insect cells closely resemble those made by mammalian cells. For the introduction of foreign DNA to insect cells, such as Drosophila S2 cells, infection with baculovirus vectors is widely used. Other insect vector systems include, for example, the expression plasmid pIZ/V5-His (InVitrogen) and other variants of the pIZ/V5 vectors encoding other tags and selectable markers. Insect cells are readily transfectable using lipofection reagents, and there are lipid-based transfection products specifically optimized for the transfection of insect cells (for example, from PanVera).

2. Host Cells Useful according to the Invention

Any cell into which a recombinant vector carrying an R. reniformis GFP or variant thereof may be introduced and wherein the vector is permitted to drive the expression of the GFP or GFP variant sequence is useful according to the invention. That is, because of the wide variety of uses for the GFP molecules of the invention, any cell in which a GFP molecule of the invention may be expressed and preferably detected is a suitable host. Vectors suitable for the introduction of GFP-encoding sequences to host cells from a variety of different organisms, both prokaryotic and eukaryotic, are described herein above or known to those skilled in the art.

Host cells may be prokaryotic, such as any of a number of bacterial strains, or may be eukaryotic, such as yeast or other fungal cells, insect or amphibian cells, or mammalian cells including, for example, rodent, simian or human cells. Cells expressing GFPs of the invention may be primary cultured cells, for example, primary human fibroblasts or keratinocytes, or may be an established cell line, such as NIH3T3, 293T or CHO cells. Further, mammalian cells useful for expression of GFPs of the invention may be phenotypically normal or oncogenically transformed. It is assumed that one skilled in the art can readily establish and maintain a chosen host cell type in culture.

3. Introduction of GFP-Encoding Vectors to Host Cells

GFP-encoding vectors may be introduced to selected host cells by any of a number of suitable methods known to those skilled in the art. For example, GFP constructs may be introduced to appropriate bacterial cells by infection, in the case of E. coli bacteriophage vector particles such as lambda or M13, or by any of a number of transformation methods for plasmid vectors or for bacteriophage DNA. For example, standard calcium-chloride-mediated bacterial transformation is still commonly used to introduce naked DNA to bacteria (Sambrook et al., 1989, Molecular Cloning, A Laboratory Manual, Cold Spring Harbor Laboratory Press, Cold Spring Harbor, N.Y.), but electroporation may also be used (Ausubel et al., 1989, supra).

For the introduction of GFP-encoding constructs to yeast or other fungal cells, chemical transformation methods are generally used (e.g. as described by Rose et al., 1990, Methods in Yeast Genetics, Cold Spring Harbor Laboratory Press, Cold Spring Harbor, N.Y.). For transformation of S. cerevisiae, for example, the cells are treated with lithium acetate to achieve transformation efficiencies of approximately 10⁴ colony-forming units (transformed cells)/μg of DNA. Transformed cells are then isolated on selective media appropriate to the selectable marker used. Alternatively, or in addition, plates or filters lifted from plates may be scanned for GFP fluorescence to identify transformed clones.

For the introduction of R. reniformis GFP-encoding vectors to mammalian cells, the method used will depend upon the form of the vector. For plasmid vectors, DNA encoding R. reniformis GFP or variants thereof may be introduced by any of a number of transfection methods, including, for example, lipid-mediated transfection (“lipofection”), DEAE-dextran-mediated transfection, electroporation or calcium phosphate precipitation. These methods are detailed, for example, in Ausubel et al., 1989, supra.

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

For the introduction of R. reniformis GFP-encoding vectors to insect cells, such as Drosophila Schneider 2 cells (S2) cells, Sf9 or Sf21cells, transfection is also performed by lipofection.

Following transfection with an R. reniformis GFP-encoding vector of the invention, eukaryotic (preferably, but not necessarily mammalian) cells successfully incorporating the construct (intra- or extrachromosomally) may be selected, as noted above, by either treatment of the transfected population with a selection agent, such as an antibiotic whose resistance gene is encoded by the vector, or by direct screening using, for example, FACS of the cell population or fluorescence scanning of adherent cultures. Frequently, both types of screening may be used, wherein a negative selection is used to enrich for cells taking up the construct and FACS or fluorescence scanning is used to further enrich for cells expressing GFPs or to identify specific clones of cells, respectively. For example, a negative selection with the neomycin analog G418 (Life Technologies, Inc.) may be used to identify cells that have received the vector, and fluorescence scanning may be used to identify those cells or clones of cells that express the R. reniformis GFP or GFP variant to the greatest extent.

4. Preparation of Antibodies Reactive with R. reniformis GFP

Antibodies that bind to a GFP polypeptide encoded by a polynucleotide of the invention are useful, for example, in protein purification and in protein association assays. An antibody useful in the invention may comprise a whole antibody, an antibody fragment, a polyfunctional antibody aggregate, or in general a substance comprising one or more specific binding sites from an antibody. The antibody fragment may be a fragment such as an Fv, Fab or F(ab′)₂ fragment or a derivative thereof, such as a single chain Fv fragment. The antibody or antibody fragment may be non-recombinant, recombinant or humanized. The antibody may be of an immunoglobulin isotype, e.g., IgG, IgM, and so forth. In addition, an aggregate, polymer, derivative and conjugate of an immunoglobulin or a fragment thereof can be used where appropriate.

GFP-derived peptides used to induce specific antibodies preferably have an amino acid sequence consisting of at least five amino acids and more conveniently at least ten amino acids. It is advantageous for such peptides to be identical to a region of the natural R. reniformis GFP protein or variant thereof, and they may even contain the entire amino acid sequence of R. reniformis GFP (e.g., SEQ ID NO: 2) or a variant thereof.

For the production of antibodies, various hosts including goats, rabbits, rats, mice, etc., may be immunized by injection with peptides or polypeptides having sequences derived from the GFP polypeptides of the invention. Depending on the host species, various adjuvants may be used to increase the immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol.

To generate polyclonal antibodies, the antigen (i.e., an R. reniformis GFP polypeptide, variant thereof, or peptide fragment derived therefrom) may be conjugated to a conventional carrier in order to increase its immunogenicity, and an antiserum to the peptide-carrier conjugate raised. Short stretches of amino acids corresponding to a GFP polypeptide of the invention may be fused, either by expression as a fusion product or by chemical linkage, with amino acids from another protein such as keyhole limpet hemocyanin or GST, with antibodies then being raised against the chimeric molecule. Coupling of a peptide to a carrier protein and immunizations may be performed as described in Dymecki et al., 1992, J. Biol. Chem., 267:4815. The serum can be titered against polypeptide antigen by ELISA or alternatively by dot or spot blotting (Boersma & Van Leeuwen, 1994, J. Neurosci. Methods, 51:317). A useful serum will react strongly with the appropriate peptides by ELISA, for example, following the procedures of Green et al., 1982, Cell, 28:477.

Techniques for preparing monoclonal antibodies are well known, and monoclonal antibodies may be prepared using an antigen, preferably bound to a carrier, as described by Arnheiter et al., 1981, Nature, 294:278. Monoclonal antibodies are typically obtained from hybridoma tissue cultures or from ascites fluid obtained from animals into which the hybridoma tissue was introduced. Monoclonal antibody-producing hybridomas (or polyclonal sera) can be screened for antibody binding to the target protein according to methods known in the art.

5. Variants of R. reniformis GFP according to the Invention

The invention provides methods of identifying variant R. reniformis GFPs that are even better suited, for example, for use in methods employing FRET or for FACS analysis than the wild-type R. reniformis GFP of amino acid sequence SEQ ID NO: 2, encoded by the polynucleotide of SEQ ID NO: 1. The wild-type GFP isolated directly from R. reniformis organisms has 3-6-fold higher quantum yield than A. victoria GFP. As shown herein in Example 4, the R. reniformis GFP polypeptide produced in mammalian cells from recombinant nucleic acid sequences of the invention has spectral characteristics nearly indistinguishable from the native polypeptide, i.e., the recombinant R. reniformis GFP of the invention is 3-6 fold brighter than that of A. victoria wild-type GFP expressed in the same cell type and has excitation and emission spectra similar to the natural R. reniformis GFP protein. However, even with the improved brightness of the recombinantly produced R. reniformis GFP over A. victoria GFP, the identification of R. reniformis GFP variants with enhanced brightness is desirable.

In addition to R. reniformis GFP variants with increased brightness, other modifications are also of interest. For example, variants exhibiting shifts in either excitation or emission spectra or both are useful since they allow the monitoring of the location or level of more than one polypeptide in the same cell through simple fluorescence measurements. Also, GFP variants with, for example, an excitation spectrum that is overlapped by the emission spectrum of another GFP (wild-type or variant) can be useful for FRET-based assays. Alternatively, GFP variants whose spectral characteristics are responsive to environmental changes, such as pH or oxidation/reduction status or are responsive to changes in phosphorylation status are useful in studies of such intracellular or even extracellular changes.

a. Mutagenesis Methods Useful according to the Invention

Modifications to the R. reniformis GFP coding sequences may be either random or targeted. In either case, selection involves monitoring individual clones for the desired modified characteristic, be it enhanced fluorescence relative to wild-type R. reniformis GFP, a spectral shift, or other modification.

Many random and site-directed mutagenesis methods are known in the art, and any of them that generate modifications to the R. reniformis GFP coding sequence of SEQ ID NO: 1 are applicable to generate variant GFPs of the invention. Several examples of both random and site-directed mutagenesis are described below.

Random Mutagenesis

Chemical mutagenesis using, for example, nitrous acid, permanganate or formic acid may be used to generate random mutations essentially as described by Meyer et al., 1985, Science 229: 242, which is incorporated herein in its entirety by reference. When following the Meyer et al. method, a mutated population of single-stranded R. reniformis GFP fragments is generated that is then amplified using the PCR primers used herein above for amplification of wild-type R. reniformis GFP. The amplification products, bearing random mutations, are cloned into an appropriate vector and transformed into bacteria, and colonies are screened for altered fluorescence characteristics relative to wild-type R. reniformis GFP either expressed from the same vector in the same bacterial strain or purified.

An alternative to chemical mutagenesis for the generation of random mutants is the use of a mutagenic bacterial strain, such as the XL1-Red E. coli strain (Stratagene), which is deficient in DNA polymerase proofreading activity and DNA repair machinery. A plasmid introduced to this or a similar strain of bacteria becomes mutated during cell division. When using a mutagenic bacterial strain such as XL1-Red, plasmids containing the GFP sequence to be mutagenized (i.e., SEQ ID NO: 1) are transformed into the mutagenic bacteria and propagated for about two days (shorter or longer, depending upon the desired degree of mutagenesis). The randomly mutated plasmids are isolated from the culture using standard methods and re-transformed into non-mutagenic bacteria (e.g., E. coli strain DH5; Life Technologies, Inc.), which are plated to achieve individual colonies. The colonies are then screened for the desired altered fluorescence characteristic relative to colonies expressing wild-type R. reniformis from the same plasmid in the same bacterial strain.

Another example of a method for random mutagenesis is the so-called “error-prone PCR method”. As the name implies, the method amplifies a given sequence under conditions in which the DNA polymerase does not support high fidelity incorporation. The conditions encouraging error-prone incorporation for different DNA polymerases vary, however one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase. As with the other methods, mutagenized sequences are inserted into an appropriate vector, transformed into bacteria and screened for the desired characteristics.

Site-Directed or Targeted Mutagenesis

There are a number of site-directed mutagenesis methods known in the art which allow one to mutate a particular site or region in a straightforward manner. These methods are embodied in a number of kits available commercially for the performance of site-directed mutagenesis, including both conventional and PCR-based methods. Examples include the EXSITE™ PCR-based site-directed mutagenesis kit available from Stratagene (Catalog No. 200502; PCR based) and the QUIKCHANGE™ site-directed mutagenesis kit from Stratagene (Catalog No. 200518; PCR based), and the CHAMELEON™ double-stranded site-directed mutagenesis kit, also from Stratagene (Catalog No. 200509).

Older methods of site-directed mutagenesis known in the art relied upon sub-cloning of the sequence to be mutated into a vector, such as an M13 bacteriophage vector, that allows the isolation of single-stranded DNA template. In these methods one annealed a mutagenic primer (i.e., a primer capable of annealing to the site to be mutated but bearing one or more mismatched nucleotides at the site to be mutated) to the single-stranded template and then polymerized the complement of the template starting from the 3′ end of the mutagenic primer. The resulting duplexes were then transformed into host bacteria and plaques were screened for the desired mutation.

More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

The protocol described below accommodates these considerations through the following steps. First, the template concentration used is approximately 1000-fold higher than that used in conventional PCR reactions, allowing a reduction in the number of cycles from 25-30 down to 5-10 without dramatically reducing product yield. Second, the restriction endonuclease DpnI (recognition target sequence: 5-Gm6ATC-3, where the A residue is methylated) is used to select against parental DNA, since most common strains of E. coli Dam methylate their DNA at the sequence 5′-GATC-3′. Third, Taq Extender is used in the PCR mix in order to increase the proportion of long (i.e., full plasmid length) PCR products. Finally, Pfu DNA polymerase is used to polish the ends of the PCR product prior to intramolecular ligation using T4 DNA ligase. The method is described in detail as follows:

PCR-based Site Directed Mutagenesis

Plasmid template DNA (approximately 0.5 pmole) is added to a PCR cocktail containing: 1× mutagenesis buffer (20 mM Tris HCl, pH 7.5; 8 mM MgCl₂; 40 ug/ml BSA); 12-20 pmole of each primer (one of skill in the art may design a mutagenic primer as necessary, giving consideration to those factors such as base composition, primer length and intended buffer salt concentrations that affect the annealing characteristics of oligonucleotide primers; one primer must contain the desired mutation, and one (the same or the other) must contain a 5′ phosphate to facilitate later ligation), 250 uM each dNTP, 2.5 U Taq DNA polymerase, and 2.5 U of Taq Extender (Available from Stratagene; See Nielson et al. (1994) Strategies 7: 27, and U.S. Pat. No. 5,556,772). The PCR cycling is performed as follows: 1 cycle of 4 min at 94° C., 2 min at 50° C. and 2 min at 72° C.; followed by 5-10 cycles of 1 min at 94° C., 2 min at 54° C. and 1 min at 72° C. The parental template DNA and the linear, PCR-generated DNA incorporating the mutagenic primer are treated with DpnI (10 U) and Pfu DNA polymerase (2.5U). This results in the DpnI digestion of the in vivo methylated parental template and hybrid DNA and the removal, by Pfu DNA polymerase, of the non-template-directed Taq DNA polymerase-extended base(s) on the linear PCR product. The reaction is incubated at 37° C. for 30 min and then transferred to 72° C. for an additional 30 min. Mutagenesis buffer (115 ul of 1×) containing 0.5 mM ATP is added to the DpnI-digested, Pfu DNA polymerase-polished PCR products. The solution is mixed and 10 ul are removed to a new microfuge tube and T4 DNA ligase (2-4 U) is added. The ligation is incubated for greater than 60 min at 37° C. Finally, the treated solution is transformed into competent E. coli according to standard methods.

Limited Random Mutagenesis

A subcategory of site-directed mutagenesis involves the use of randomized oligonucleotides to introduce random mutations into a limited region of a given sequence (this will be referred to as “limited random mutagenesis”). This is particularly useful when one wishes to mutate every base within, for example, a region encoding a hexapeptide. Generally, the oligonucleotides used for this type of approach have a stretch of constant nucleotides exactly complementary to a region on either side of and immediately adjacent to the region to be mutated, linked by a randomized or partially randomized oligonucleotide sequence corresponding to the sequence to be mutated. One of the constant sequences flanking the mutagenic region should have a restriction site to facilitate the replacement of wild-type sequence with the mutagenized sequence following mutagenesis. Ideally, such a restriction site is naturally present adjacent to the region to be mutated, but one skilled in the art may also introduce restriction sites through silent mutations, without altering the coding sequence (see, for example, the list of restriction sites that may be introduced by silent mutagenesis in the New England Biolabs (NEB) catalog appendices, specifically at pages 282-283 of the 1998/1999 NEB catalog).

In the limited random mutagenesis method, mutagenic oligonucleotides as described above are used, along with a selected partner primer, and a wild type, or even previously mutated, recombinant R. reniformis GFP construct template (wild-type, or, alternatively, previously altered) to PCR amplify a pool of fragments, all randomly or semi-randomly mutated at the desired sites. The partner primer is selected so that it is either 5′ or 3′ of the mutagenized stretch of nucleotides, and should have either a naturally occurring restriction site or an engineered restriction site that does not alter GFP coding sequences, to permit the replacement of the wild-type with the mutated sequences. Conveniently, the partner primer may bind in the vector sequences immediately 5′ or 3′ of the GFP coding sequence. The amplified pool of mutated fragments is cleaved with the restriction enzymes recognizing the respective sites in the mutagenic and partner primers, and the pool is ligated into a similarly cleaved recombinant vector comprising the GFP coding sequences (either 5′ of or 3′ of the mutagenized site) not amplified during the mutagenic step, to generate a pool of full length GFP coding sequences randomly or semi-randomly mutated only over the selected stretch of nucleotides.

The mutations in the limited random mutagenesis approach are referred to as “random or semi-random” because the mutagenic sequences do not necessarily have to be completely random. One of skill in the art will recognize, for example, that it is possible to vary one, two, or all three nucleotides in a codon with different results as far as the range of possible changes to the peptide sequence encoded, from no change (often possible in the third or “wobble” nucleotide) to limited change (changes affecting the middle and or third nucleotide only) to completely random change (changes affecting all three nucleotides of the codon). Therefore, by maintaining some nucleotides constant within the mutagenized region and allowing others to vary (either over all four possible nucleotides or over one or more subsets of them), the characteristics of the mutagenized region may be controlled. Sequences mutagenized in such a manner would be “semi-randomly” mutagenized. Following the cloning of the mutated pool of R. reniformis GFP vectors using the limited random mutagenesis method, or its equivalent, the mutated pool is transformed into bacteria, expression is induced, and the clones are screened for the desired altered characteristic.

b. Purification of R. reniformis GFP or Variants Thereof

If necessary, R. reniformis GFP is purified from R. reniformis organisms as described by Ward and Cormier (1979, J. Biol. Chem. 254: 781-788) and by Matthews et al. (1977, Biochemistry 16: 85-91), the contents of both of which are herein incorporated by reference. Similar procedures may be applied by one of skill in the art to bacterially expressed R. reniformis GFP or variants thereof following freeze-thaw lysis and preparation of a clarified lysate by centrifugation at 14,000× g. Briefly, the methods employed by Matthews et al. and Ward and Cormier involve successive chromatography over DEAE-cellulose, Sephadex G-100, and DTNB (5,5′-dithiobis(2-nitrobenzoic acid))-Sepharose columns, and dialysis against 1 mM Tris (pH 8.0), 0.1 mM EDTA. The dialyzed fractions containing GFP (identified by fluorescence) are then acid treated to precipitate contaminants, followed by neutralization of the supernatant, which is lyophilized. Low salt (10 mM to 1 mM initially) and pH ranging from 7.5 to 8.5 are critical to maintaining activity upon lyophilization. The lyophilized sample is resuspended in water, immediately centrifuged to remove less-soluble contaminants and applied to a Sephadex G-75 column. GFP is eluted in 1.0 mM Tris (pH 8.0), 0.1 mM EDTA. Samples are concentrated by partial lyophilization and dialyzed against 5 mM sodium acetate, 5 mM imidazole, 1 mM EDTA, pH 7.5, followed by chromatography over a DEAE-BioGel-A column equilibrated in the same dialysis buffer. GFP is eluted with a continuous acidic gradient from pH 6.0 to 4.9 in the same acetate/imidizole buffer. Following dialysis of GFP-containing fractions against 1.0 mM Tris-HCl, 0.1 mM EDTA, pH 8.0, the sample is partially lyophilized to concentrate and passed over a Sephadex G-75 (Superfine) column. The GFP-containing fractions are then loaded onto a DEAE-BioGel A column in Tris/EDTA buffer at pH 8.0, followed by elution in a continuous alkaline gradient from pH 8.5 to 10.5 formed with 20 mM glycine, 5 mM Tris-HCl and 5 mM EDTA. GFP-containing fractions contain essentially homogeneous R. reniformis GFP.

In screening applications requiring less pure GFP preparations, recombinant R. reniformis or variants thereof can be purified from bacteria as follows. Bacteria transformed with a recombinant GFP-encoding vector of the invention are grown in Luria-Bertani medium containing the appropriate selective antibiotic (e.g., ampicillin at 50 μg/ml). If the vector permits, recombinant polypeptide expression is induced by the addition of the appropriate inducer (e.g., IPTG at 1 mM). Bacteria are harvested by centrifugation and lysed by freeze-thaw of the cell pellet. Debris is removed by centrifugation at 14,000× g, and the supernatant is loaded onto a Sephadex G-75 (Pharmacia, Piscataway, N.J.) column equilibrated with 10 mM phosphate buffered saline, pH 7.0. Fractions containing GFP are identified by fluorescence emission at 506 nm when excited by 500 nm light, or by excitation and emission over a range of spectra when purifying GFP variants with altered spectral characteristics.

c. Modifications to R. reniformis GFP Useful according to the Invention

The R. reniformis chromophoric center is comprised of amino acids 64-69 of the wild-type polypeptide, which has the sequence FQYGNR. Mutation of this amino acid sequence at one or more positions, using for example, standard site-directed or limited random mutagenesis or its equivalent, can give rise to R. reniformis variants exhibiting enhanced fluorescence intensity or shifted spectral characteristics. Changes at sites outside of the chromophoric center may also be affect the fluorescence properties of the polypeptide. For example, because R. reniformis lives at a temperature significantly below 37° C., mutations that stabilize the folded fluorescent form of the polypeptide at 37° C. may enhance the fluorescence of the polypeptide in human or mammalian cell culture, or in bacterial cultures, for that matter. Further, while the chemical nature of the R. reniformis GFP chromophore is nearly identical to that of the A. victoria GFP chromophore (Ward et al., 1980, Photochem. Photobiol. 31: 611-615), the fluorescence characteristics, including intensity and spectra are quite different. This indicates that modifications outside of the chromophoric center will likely have an impact on fluorescence characteristics.

In addition to modifications that change the coding sequence of wild-type R. reniformis GFP, the nucleic acid sequence encoding the polypeptide may be modified to enhance its expression in mammalian or human cells. The codon usage of R. reniformis is optimal for expression in R. reniformis, but not for expression in mammalian or human systems. Therefore, the adaptation of the sequence isolated from the sea pansy for expression in higher eukaryotes involves the modification of specific codons to change those less favored in mammalian or human systems to those more commonly used in these systems. This so-called “humanization” is accomplished by site-directed mutagenesis of the less favored codons as described herein or as known in the art. Similar modifications of the A. victoria GFP coding sequences are described in U.S. Pat. No. 5,874,304. The preferred codons for human gene expression are listed in Table 1. The codons in the table are arranged from left to right in descending order of relative use in human genes. Consideration of the codons in R. reniformis GFP (SEQ ID NO: 1) relative to those favored in human genes allows one of skill in the art to identify which codons to modify in the R. reniformis GFP gene to achieve more efficient expression in human or mammalian cells. In particular, those codons underlined in the table are almost never used in known human genes and, if found in the R. reniformis sequence would therefore represent the most important codons to modify for enhanced expression efficiency in mammalian or human cells.

TABLE 1
PREFERRED DNA CODONS FOR HUMAN USE

| | |
|---|---|
| Amino Acids | Codons Preferred in Human Genes |

| | | | |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | E | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG TTG CTT CTA TTA |
| Methionine | Met | M | ATG |
| Asparagine | Asn | N | AAC AAT |
| Proline | Pro | P | CCC CCT CCA CCG |
| Glutamine | Gln | Q | CAG CAA |
| Arginine | Arg | R | CGC AGG CGG AGA CGA CGT |
| Serine | Ser | S | AGC TCC TCT AGT TCA TCG |
| Threonine | Thr | T | ACC ACA ACT ACG |
| Valine | Val | V | GTG GTC GTT GTA |
| Tryprophan | Trp | W | TGG |
| Tyrosine | Tyr | Y | TAC TAT |

The codons at the left represent those most preferred for use in human genes, with human usage decreasing towards the right. Underlined codons are almost never used in human genes.

6. Screening For R. reniformis GFP Mutants with Altered Fluorescence Characteristics or Altered Traits

One method of screening for altered fluorescence characteristics involves lifting single bacterial colonies transformed with a mutated GFP sequence from a plate onto a support, such as 0.45 μm pore size nitrocellulose membranes (Schleicher & Schuell, Keene, N.H.), placing the membranes onto fresh agar/medium plates (e.g., LB agar containing 50 μg/ml ampicillin, 1 mM IPTG for a vector containing amp^{r} and laci repressor genes, and a lac operator upstream of the R. reniformis GFP coding region), bacteria-side up, and allowing colonies to grow on the membrane. The membranes are then scanned for fluorescence characteristics of the colonies. Scanning may be performed under illumination with monochromatice light, for example as generated by passing light from a 150 W Xenon lamp (Xenon Corp., Woburn, Mass.) through interference filters appropriate for the desired excitation wavelengths (filters available, for example, from CVI Laser Corp., Albuquerque, N.Mex.). Emissions from the illuminated colonies may be observed through, for example, a Schott KV500 filter, which has a 500 nm wavelength cutoff. The same methods of screening mutants for altered fluorescence characteristics are applicable regardless of whether mutagenesis is random or targeted.

Alternative fluorescence scanning equipment includes a scanning polychromatic light source (such as a fast monochromator from T.I.L.L. Photonics, Munich, Germany) and an integrating RGB color camera (such as the Photonic Science Color Cool View). Following multi-wavelength excitation scanning, images captured by the integrating color camera may be subjected to image analysis to determine the actual color of the emitted light using software such as Spec R4 (Signal Analytics Corp., Vienna, Va., USA).

With many of the altered characteristics (e.g., fluorescence intensity, thermal stability or spectral characteristics) being screened for, bacteria or eukaryotic (e.g., yeast or mammalian) cells expressing the mutated form may first be screened relative to control cells expressing the wild-type form, followed if necessary by characterization of either clarified lysates or purified polypeptides from those colonies selected by the cellular screen. For other altered characteristics (e.g., pH sensitivity or phosphorylation-dependent alteration of fluorescence), purified polypeptides or at least clarified bacterial or eukaryotic cell lysates may be necessary for screening. Where necessary, clarified lysate preparation and/or purification is/are achieved according to methods described herein or known in the art. Ultimately, purified mutated or altered GFP polypeptides can be compared to wild-type R. reniformis GFP (native or recombinant) with regard to the characteristic one desires to modify. When screening for mutants of R. reniformis GFP with altered fluorescence intensity or brightness according to the invention, one looks for fluorescence that is at least two times more intense or bright than the fluorescence of wild-type R. reniformis GFP (either isolated from R. reniformis or expressed from a recombinant vector construct of the invention), and up to 3 times, 5 times, 10 times, 20 times, 50 times or even 100 or more times as intense or bright as the same molar amount of wild-type R. renifirmis GFP.

When screening for R. reniformis GFP mutants with altered spectral characteristics, one looks for GFP polypeptides that exhibit excitation or emission spectra that are distinguishable or detectably distinct from those of the wild-type GFP polypeptide. By distinguishable or detectably distinct is meant that standard filter sets allow either the excitation of one form without excitation of the other form, or similarly, that standard filter sets allow the distinction of the emission from one form from the other. Generally, distinguishable excitation or emission spectra have peaks that vary by more than 1 nm, and preferably vary by more than 2, 3, 4, 5, 10 or more nm. The peaks of distinguishable spectra are also preferably narrow, covering a range of about 5 nm or less, 7 nm or less, 10 nm or less, 15 nm or less, 20 nm or less, 50 nm or less, or 100 nm or less. The maximum allowable breadth of a peak that is considered distinguishable is directly related to how much the peak maximum varies from the maximum of the peak it is being distinguished from. In other words, the larger the variance between the peak wavelengths of two fluorescent polypeptides, the broader the peaks may be and still be distinguishable. Conversely, the lower the variance between the centers of the peaks, the narrower the peaks must be to be distinguishable.

Particularly preferred spectral shifts are shifts in emission spectra that are not accompanied by distinguishable shifts in excitation spectra. Such a shift permits the excitation of two or more different GFPs with light of the same wavelength (or same range of excitation wavelengths) yet also permits distinction of the fluorescence of two or more GFPs based on the different emission wavelengths.

Other preferred spectral shifts include those that render the R. reniformis GFP capable of FRET as either a donor or an acceptor fluoroprotein. For example, a spectral alteration that changes the excitation spectrum of a first fluorescent polypeptide so that it overlaps the emission spectrum of a second fluorescent polypeptide will define a pair of fluorescent polypeptides capable of FRET. It is preferred, although not necessary that both the first and second fluorescent polypeptides be GFP polypeptides; if a non-GFP fluorescent polypeptide is a donor or acceptor for FRET, it is preferred that a polynucleotide sequence for that fluorescent polypeptide is known.

If both fluorescent polypeptides of a FRET pair are R. reniformis GFP polypeptides, one or both polypeptides may be altered. That is, one may be wild-type R. reniformis GFP and the other may be altered, or both GFPs of the FRET pair may be altered. In the case in which wild-type R. reniformis GFP is a member of the pair, it may be either the donor or the acceptor member of the pair.

Another altered characteristic that may enhance the usefulness of the R. reniformis GFP polypeptides of the invention is altered stability of the polypeptide in vivo. As mentioned above, modifications that alter the folded stability of the polypeptide's fluorophore center can alter the fluorescence intensity of the polypeptide. However, modifications that increase or reduce the in vivo or in vitro half-life of the entire GFP polypeptide, i.e., modifications that affect polypeptide turnover or degradation are also useful. For example, increased stability can enhance the detection of the modified R. reniformis GFP by allowing a larger steady-state pool of GFP to accumulate at a given expression rate. Importantly, there is also usefulness for R. reniformis GFP polypeptide variants with reduced in vivo or in vitro stability. For example, the responsiveness of reporter assays for transcription is enhanced by reporter molecules with shorter half-lives. Generally, the shorter the biological half-life of the reporter molecule, the faster a new steady state is achieved when the transcription rate increases or decreases, enhancing the sensitivity of the assay.

II. How to Use R. reniformis GFP and Variants Thereof according to the Invention

R. reniformis GFP and variants thereof according to the invention are useful in a number of different ways. Generally, R. reniformis is useful in any process or assay that can be performed with A. victoria GFP. Further, because of its superior spectral characteristics and fluorescent intensity, wild-type R. reniformis GFP is useful in processes and assays beyond those that can be performed with A. victoria GFP. And finally, altered, modified or mutated R. reniformis is even more useful for particular applications of fluorescent marker technologies.

R. reniformis GFP or variants thereof may be used as selectable markers for the identification of cells transfected or infected with a gene transfer vector. In this aspect, cells transfected with a construct encoding GFP may be identified over a background of non-transfected or infected cells by illumination of the cells with light within the excitation spectrum and detection of fluorescent emission in the emission spectrum of the GFP.

The usefulness of R. reniformis GFP as a reporter molecule stems from properties such as ready detection, the feasibility of real-time detection in vivo, and the fact that the introduction of a substrate is not required. R. reniformis gfp genes can therefore be used to identify transformed cells (e.g., by fluorescence-activated cell sorting (FACS) or fluorescence microscopy), to measure gene expression in vitro and in vivo, to label specific cells in multicellular organisms (e.g., to study cell lineages), to label and locate fusion proteins, and to study intracellular protein trafficking. Variant R. reniformis GFPs exhibiting altered fluorescence characteristics in response to changes in, for example, pH, phosphorylation status or redox status are useful for studying changes in those parameters in vivo.

R. reniformis GFPs may also be used for standard biological applications. For example, they may be used as molecular weight markers on protein gels and Western blots, in calibration of fluorometers and FACS equipment and as a marker for micro injection into cells and tissues. In methods to produce fluorescent molecular weight markers, an R. reniformis GFP gene sequence is fused to one or more DNA sequences that encode proteins having defined amino acid sequences, and the fusion proteins are expressed from an expression vector. Expression results in the production of fluorescent proteins of defined molecular weight or weights that may be used as markers.

Preferably, purified fluorescent proteins are subjected to size-fractionation, such as by using a gel. A determination of the molecular weight of an unknown protein is then made by compiling a calibration curve from the fluorescent standards and reading the unknown molecular weight from the curve.

A. Uses of R. reniformis GFPs with Altered Emission Spectra

Amino acid replacements in R. reniformis GFP that produce different color emission spectra permit simultaneous use of multiple reporter genes. Different colored R. reniformis GFPs can be used to identify multiple cell populations in a mixed cell culture or to track multiple cell types, permitting differences in cell movement or migration to be visualized in real time without the need to add additional agents or fix or kill the cells.

Other options involving the uses of GFPs with altered emission spectra include tracking and determining the ultimate location of multiple proteins within a single cell, tissue or organism. Differential promoter analysis in which gene expression from two different promoters is determined in the same cell, tissue or organism is also permitted by GFPs with differing emission spectra, as is and FACS sorting of mixed cell populations.

In tracking proteins within a cell, the R. reniformis GFP variants are used in a manner analogous to fluorescein and rhodamine to tag interacting proteins or subunits whose association is then be monitored dynamically in intact cells by FRET. Cells are irradiated with light at the excitation wavelengths of the donor, and emission by the acceptor is monitored to indicate protein: protein interactions of tagged proteins.

The techniques that can be used with spectrally separable R. reniformis GFP derivatives are exemplified by confocal microscopy, flow cytometry, and fluorescence activated cell sorting (FACS) using modular flow, dual excitation techniques.

B. Use of R. reniformis GFP in the Identification of Transfected Cells

R. reniformis GFP may be introduced as a selectable marker to identify transfected cells from a background of non-transfected cells. Alternatively, R. reniformis GFP transfection may be used to pre-label isolated cells or a population of similar cells prior to exposing the cells to an environment in which different cell types are present. Detection of GFP in only the original cells allows the location of such cells to be determined and compared with the total population.

Cells that have been transfected with exogenous DNA can be identified with the R. reniformis GFPs of the invention. out creating a fusion protein. The method relies on the identification of cells that have received a plasmid or vector that comprises at least two transcriptional or translational units. A first unit will encode and direct expression of the desired protein, while the second unit will encode and direct expression of R. reniformis GFP or a variant thereof. Co-expression of GFP from the second transcriptional or translational unit ensures that cells containing the vector are detected and differentiated from cells that do not contain the vector.

The R. reniformis GFP sequences of the invention may also be fused to a DNA sequence encoding a selected protein in order to directly label the encoded protein with GFP. Expressing such an R. reniformis GFP fusion protein in a cell results in the production of fluorescently-tagged proteins that can be readily detected. This is useful in confirming that a protein is being produced by a chosen host cell. It also allows the location of the selected protein to be determined, whether this represents a natural location or whether the protein has been artificially targeted to another location.

C. Analysis of Transcriptional Regulatory Sequences

The R. reniformis GFP genes of the invention allow a range of transcriptional regulatory sequences to be tested for their suitability for use with a given gene, cell, or system. This applies to in vitro uses, such as in identifying a suitable transcriptional regulatory sequence for use in recombinant expression and high level protein production, as well as in vivo uses, such as in pre-clinical testing or in gene therapy in human subjects.

In order to analyze a transcriptional regulatory sequence, one must first establish a control cell or system. In the control, a positive result is established by using a known and effective promoter, such as the CMV promoter. To test a candidate transcriptional regulatory sequence, another cell or system is established in which all conditions are the same except for there being different transcriptional regulatory sequences in the expression vector or genetic construct.

After running the assay for the same period of time and under the same conditions as in the control, the GFP expression levels are determined. This allows one to make a comparison of the strength or suitability of the candidate transcriptional regulatory sequence with the standard or control transcriptional regulatory sequence.

Transcriptional regulatory sequences that can be tested in this manner also include candidate tissue-specific promoters and candidate-inducible promoters. Testing of tissue-specific promoters allows the identification of optimal transcriptional regulatory sequences for use with a given cell. Again, this is useful both in vitro and in vivo. Optimizing the combination of a given transcriptional regulatory sequence and a given cell type in recombinant expression and protein production is often necessary to ensure that the highest possible expression levels are achieved.

The GFP encoded by a regulatory sequence testing construct may optionally have a secretion signal fused to it, such that GFP secreted to the medium is detected.

The use of tissue-specific promoters and inducible promoters is particularly powerful in vivo embodiments. When used in the context of expressing a therapeutic gene in an animal, the use of such transcriptional regulatory sequences allows expression only in a given tissue or tissues, at a given site and/or under defined conditions. Achieving tissue-specific expression is particularly important in certain gene therapy applications, such as in the expression of a cytotoxic agent, as is often employed in approaches to the treatment of cancer. In expressing other therapeutic genes with a beneficial effect, rather than a cytotoxic effect, tissue-specific expression is also preferred since it can optimize the effect of the treatment. Appropriate tissue-specific and inducible transcriptional regulatory sequences are known to those of skill in the art, or, for example, described herein above.

D. Use of R. reniformis GFP in Assays for Compounds that Modulate Transcription

R. reniformis GFP and variants thereof are useful in screening assays to detect compounds that modulate transcription. In this aspect of the invention, R. reniformis GFP coding sequences are positioned downstream of a promoter that is known to be inducible by the agent that one wishes to detect. Expression of GFP in the cells will normally be silent, and is activated by exposing the cell to a composition that contains the selected agent. In using a promoter that is responsive to, for example, a lipid soluble transcriptional modulator, a toxin, a hormone, a cytokine, a growth factor or other defined molecule, the presence the particular defined molecule can be determined. For example, an estrogen-responsive regulatory sequence may be linked to GFP in order to test for the presence of estrogen in a sample.

It will be clear to one of skill in the art that any of the detection assays may be used in the context of screening for agents that inhibit, suppress or otherwise down regulate gene expression from a given transcriptional regulatory sequence. Such negative effects are detectable by decreased GFP fluorescence that results when gene expression is down-regulated in response to the presence of an inhibitory agent.

E. Use of R. reniformis GFP and Variants Thereof in FACS Analyses

Many conventional FACS methods require the use of fluorescent dyes conjugated to purified antibodies. Fusion proteins tagged with a fluorescent label are preferred over antibodies in FACS applications because the cells do not have to be incubated with the fluorescent-tagged reagent and because there is no background due to nonspecific binding of an antibody conjugate. GFP is particularly suitable for use in FACS as fluorescence is stable and species-independent and does not require any substrates or cofactors.

As with other expression embodiments, a desired protein may be directly labeled with GFP by preparing and expressing a GFP fusion protein in a cell. GFP can also be co-expressed from a second transcriptional or translational unit within the expression vector that expresses desired protein, as described above. Cells expressing the GFP-tagged protein or cells co-expressing GFP are then detected and sorted by FACS analysis. An advantage of GFP from R. reniformis is that its excitation and emission spectra are amenable to standard optics and filter sets used in FACS analyses.

F. Other Uses of R. reniformis GFP Fusion Proteins

R. reniformis GFP genes can be used as one portion of a fusion protein, allowing the location of the tagged protein to be identified. Fusions of GFP with an exogenous protein should preserve both the fluorescence of GFP and functions of the host protein, such as physiological functions and/or targeting functions.

Both the amino and carboxyl termini of GFP may be fused to virtually any desired protein to create an identifiable GFP-fusion, and fusion may be mediated by a linker sequence if necessary to preserve the function of the fusion partner.

R. reniformis GFP fusions are useful for subcellular localization studies. Localization studies have previously been carried out by subcellular fractionation and by immunofluorescence. However, these techniques can give only a static representation of the position of the protein at one instant in the cell cycle. In addition, artifacts can be introduced when cells are fixed for immunofluorescence. Using GFP to visualize proteins in living cells, which allows proteins to be followed throughout the cell cycle in an individual cell, is thus an important technique.

R. reniformis GFP can be used to analyze intracellular protein traffic in mammalian and human cells under a variety of conditions in real time. Artifacts resulting from fixing cells are avoided. In these applications, R. reniformis GFP is fused to a known protein in order to examine its sub-cellular location under different natural conditions.

### EXAMPLES

### Example 1

### Production of Infectious R. reniformis GFP Retroviruses

Virus production was carried out by co-transfecting 293T cells with 3 μg each of the vectors pGPhisD (Stratagene), pVSV-G-puro (Stratagene), and either pFB-rGFP or the vector pFB-AvGFP. The latter vector contains a copy of the A. victoria GFP gene that includes an insertion of the alanine codon GCT immediately following the methionine initiation codon to accommodate the inclusion of a Kozak consensus sequence, as well as the Ser->Thr “red shift” amino acid substition at position 65 (relative to the wt sequence). The vectors pGPhisD and pVSV-G-puro encode the viral proteins gag-pol and VSV-G, which are required in trans for production of virus.

The transfections were carried out using the MBS Transfection Kit (Stratagene), with some modifications. For each transfection, 2.5×10⁶ 293T cells were plated in a 60 mm tissue culture dish. The following day medium was aspirated and replaced with 4 ml pre-warmed DMEM supplemented with 7% MBS and 25 μM chloroquine (Sigma, St. Louis, Mo.) prior to transfection. The DNA/CaPO₄ transfection mixes were prepared according to the manufacturer's recommended protocol and added to the cells. After a 3 h incubation, the medium was replaced with 4 ml of pre-warmed complete culture medium (DMEM containing 10% Fetal Bovine Serum (FBS)) supplemented with 25 μM chloroquine and incubated for 6-7 hours. The medium was then replaced with 4 ml of pre-warmed DMEM+10% FBS. Cells were incubated overnight (12-16 hours), and medium was replaced with 3 ml pre-warmed DMEM+10% FBS, and virus was collected overnight (24 hours). The 3 ml viral supernatant was removed and filtered through a 0.45 μm filter. Supernatants were stored on ice for immediate use or frozen on dry ice and stored at −80 C.

### Example 2

### Transduction of Host Cells with R. reniformis GFP Retroviral Stocks

One day prior to transduction, NIH3T3 cells were plated in DMEM supplemented with 10% Calf Serum (CS) at 1×10⁵ cells/well in a 6 well tissue culture dish. The following day the viral supernatants were serially diluted in DMEM+10% CS to a final volume of 1.0 ml/sample, and supplemented with DEAE-Dextran (Sigma, St. Louis, Mo., catalog #D-9885) to a final concentration of 10 μg/ml. Culture medium was removed from the NIH3T3 cells and replaced with 1 ml of viral dilution. Each diluted viral sample was applied to a well containing the NIH3T3 cells, and incubated for 3 h, after which 1 ml of pre-warmed DMEM+10% CS was added to each well, and the plates were then incubated for 2 d. After 2 d the plates were washed 2× with PBS, trypsinized, pelleted by centrifugation, and resuspended in 1.0 ml PBS. Cell suspensions were stored on ice and analyzed by Fluorescence Activated Cell Sorting (FACS) within one hour. FACS analysis was performed by Cytometry Research Services, (Sorrento Valley, Calif.).

### Example 3

### Transfection of CHO Cells and Extract Preparation

CHO cells were transfected with the plasmid pFB-rGFP using Lipofectamine (BRL) according to the manufacturers recommendations. Two days following transfection, soluble protein extracts were prepared from transfected and untransfected CHO cells by first washing the cells 2× with PBS, and then subjecting the cells to three freeze-thaw cycles in 0.25 M Tris-HCl, pH 7.8. The lysates were cleared by high speed centrifugation, and the supernatants were then used for spectral analyses.

### Example 4

### Spectral Analysis of Recombinant R. reniformis GFP

Excitation and emission spectral analysis was determined using a Shimadzu RF-1501 Spectrofluorophotometer. Excitation and emission scans were performed on equal amounts of total protein prepared from transfected or untransfected CHO cells. Background fluorescence was subtracted from the scans of the GFP-containing (transfected) extract by normalization to the scans of the untransfected extracts.

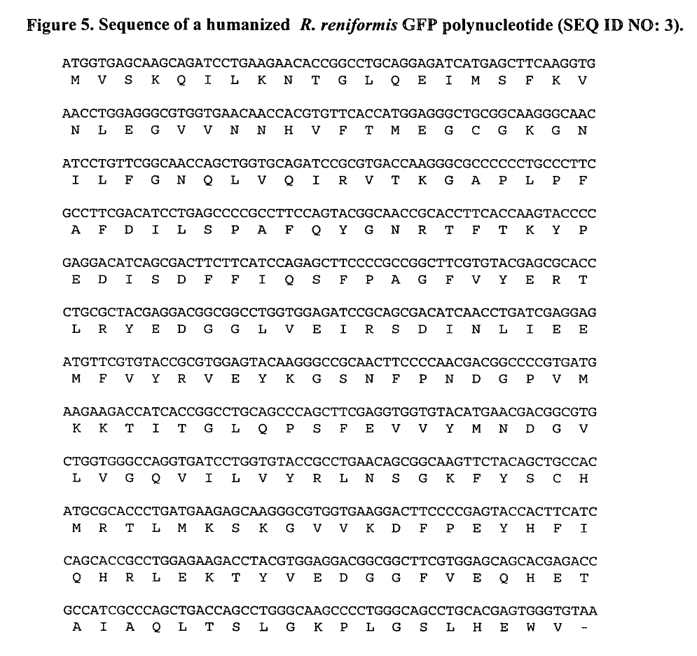

In order to compare the fluorescence profile for the cloned R. reniformis protein to that for the purified native protein, excitation and emission scans were carried out using soluble protein extracts from CHO cells transfected with the expression vector. As shown in FIG. 4, the fluorescence profile for the cloned protein is virtually identical to that reported for the native protein, with a single major excitation peak at 500 nm (compared with 498 nm for the native protein) preceded by a vibrational shoulder at approximately 470 nm, a characteristic of the native Renilla GFPs. The emission spectra show a single peak at 506 nm for the cloned protein, compared with the reported maximum of 509 nm for the native protein.

### Example 5

### Preparation of a Humanized R. reniformis GFP Polynucleotide

Expression of ectopic genes in the cells of a particular species is very often enhanced if the polynucleotide sequence of the gene is altered to make use of codons that are preferred in highly expressed genes endogenous to the cell type of choice. For example, the “humanization” of the red-shifted Aequorea GFP resulted in a dramatic enhancement of the level of fluorescence when expressed in mammalian cells (Yang, T.-T. et. al [1996] Nucl. Acids Res. 24[22]:4592-4593).

The inventors have altered 166 of the gene's 238 codons such that all of the codons in the resulting gene are biased for high expression in human cells. The codon changes were based upon the human codon usage preferences described in Haas et al., 1996, Curr Biol. 6[3]: 315-4593. The codon usage preferences shown in Table 1 are equivalent to those in the Haas reference.

Cell culture. 293, 293T and CHO cells were maintained at 37° C. at 5% CO₂ in Dulbecco's Modifed Eagle Medium (DMEM) containing 10% Fetal Bovine Serum (Gemini Bio-Products, Inc.) and 1% glutamine.

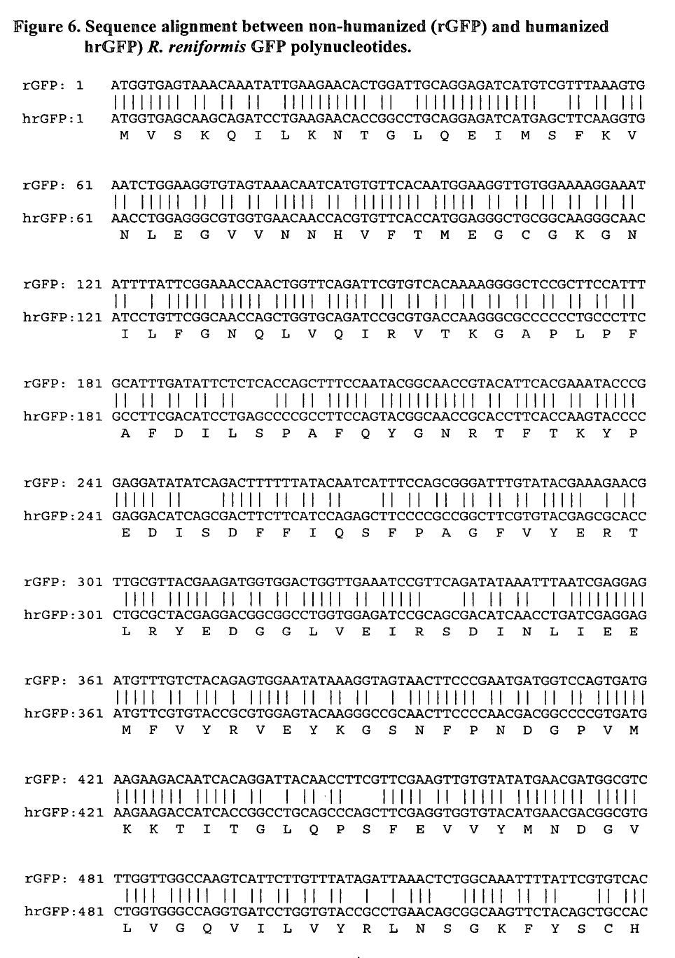

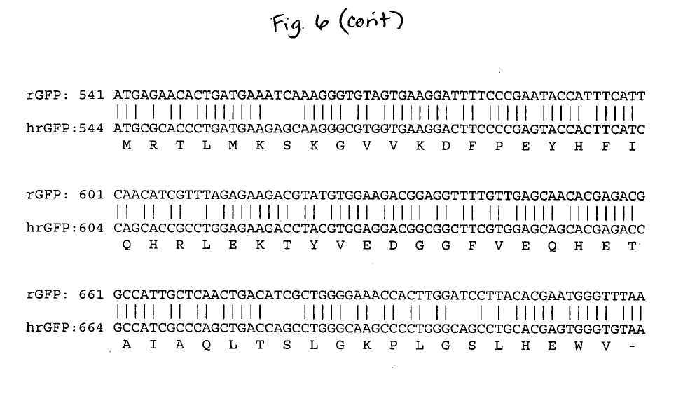

Construction of the hrGFP gene. The humanized recombinant GFP (hrGFP) nucleotide sequence was altered according to Haas, J. et. al., 1996, Curr. Biol. 6[3]:315-324, such that all the codons were selected based on their prevalence in genes that are highly expressed in human cells. The sequence is set forth in SEQ ID NO: 3 (see FIG. 5). FIG. 6 shows a sequence alignment of the non-humanized recombinant R. reniformis GFP (SEQ ID NO: 1) and humanized R. reniformis GFP polynucleotide sequences. The humanized gene was constructed by synthesizing a set of complementary, overlapping oligonucleotides which were annealed, ligated and subcloned. Both strands were completely sequenced, and mutations were corrected using the QuickChange kit (Stratagene). The PCR fragment was digested to completion with EcoR I and Xho I and inserted between the EcoR I and Xho I sites of the retroviral expression vector pFB (Stratagene) to create the vector pFB-hrGFP. This vector was used for further analysis of the humanized gene.

Virus production. Virus production was carried out by co-transfecting 293T cells with 3 μg each of the vectors pGPhisD (Stratagene), pVSV-G-puro (Stratagene), and either pFB-hrGFP or the vector pFB-EGFP. The latter vector contains a copy of the fully humanized, redshifted A. victoria GFP gene (EGFP). The vectors pGPhisD and pVSV-G-puro encode the viral proteins gag-pol and VSV-G, which are required in trans for production of virus. The transfections were carried out using the MBS Transfection Kit (Stratagene), with some modifications. For each transfection, 2.5×10⁶ 293T cells were plated in a 60 mm tissue culture dish. The following day medium was aspirated and replaced with 4 ml pre-warmed DMEM supplemented with 7% MBS and 25 μM chloroquine (Sigma, St. Louis, Mo.) prior to transfection. The DNA/CaPO₄ transfection mixes were prepared according to the manufacture's recommended protocol and added to the cells. After a 3 h incubation, the medium was replaced with 4 ml of pre-warmed complete culture medium (DMEM containing 10% FBS) supplemented with 25 μM chloroquine and incubated for 6-7 hours. The medium was then replaced with 4 ml pre-warmed DMEM+10% FBS. Cells were incubated overnight (12-16 hours), and medium was replaced with 3 ml pre-warmed DMEM+10% FBS, and virus was collected overnight (24 hours). The 3 ml viral supernatant was removed and filtered through a 0.45 μm filter. Supernatants were stored on ice for immediate use or frozen on dry ice and stored at −80° C.

### Example 6

### Evaluation of the Expression of R. reniformis GFP from a Humanized Polynucleotide Sequence

The humanized R. reniformis GFP coding sequence described in Example 5 has been tested for expression in several human, rodent and monkey cell lines. Fluoresence levels have been found to be substantially higher for the humanized rGFP (hrGFP) gene compared with that for rGFP. In a direct comparison between cell populations harboring single copy proviral expression cassettes encoding either hrGFP or the humanized, red-shifted Aequorea GFP (EGFP), we found relative fluorescence intensity to be comparable between the two genes.

Viral Transduction. One day prior to transduction, 293 cells (human) or CHO cells (hamster) were plated in DMEM supplemented with 10% FBS at 1×10⁵ cells/well in a 6 well tissue culture dish. The following day the viral supernatants were serially diluted in DMEM+10% FBS to a final volume of 1.0 ml/sample, and supplemented with DEAE-Dextran (Sigma, St. Louis, Mo., catalog #D-9885) to a final concentration of 10 μg/ml. Culture medium was removed from the target cells and replaced with 1 ml of viral dilution. Each diluted viral sample was applied to a well containing the target cells, and incubated for 3 h, after which 1 ml of pre-warmed DMEM+10% FBS was added to each well, and the plates were then incubated for 2 d. After 2 d the plates were washed 2× with PBS, trypsinized, pelleted by centrifugation, and resuspended in 1.0 ml PBS. Cell suspensions were stored on ice and analyzed by Fluorescence Activated Cell Sorting (FACS) within one hour. FACS analysis was performed by Cytometry Research Services, (Sorrento Valley, Calif.).

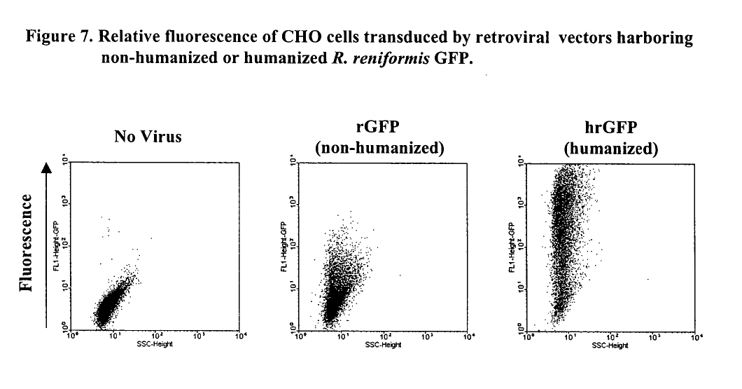

Comparison of rGFP and hrGFP expression in vivo. To determine whether the sequence alterations introduced into the R. reniformis GFP gene resulted in enhanced expression, the hrGFP coding sequence was inserted into the vector pFB, and the resulting vector pFB-hrGFP was transfected side-by-side with the parental vector pFB-rGFP gene into CHO cells. Visual inspection of the transfected cells by fluorescence microscopy (excitation 450-490 nm; emission 520 nm) revealed a dramatic enhancement of fluorescence for the hrGFP gene compared with rGFP (data not shown). CHO cells were next infected with virus derived from the two vectors at equivalent multiplicities of infection (MOI), and two days following infection the transduced cells were analyzed by fluorescence-activated cell sorting (FACS; excitation 488 nm, emission 515-545 nm). As the results in FIG. 7 indicate, the majority of the cell population transduced with pFB-hrGFP fluoresces approximately 2-3 orders of magnitude brighter than cells harboring pFB-rGFP.

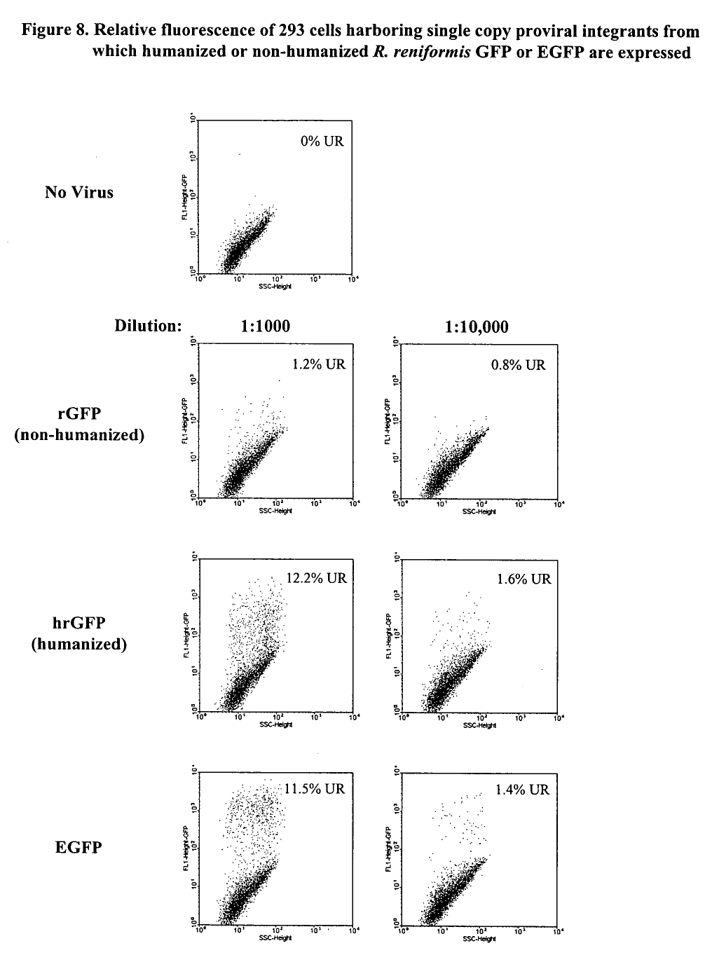

The relative fluorescence was compared from cells harboring single-copy proviral integrants encoding rGFP, hrGFP or EGFP. 293 cells were infected at low MOI, and two days post-infection the fluoresence levels were analysed by FACS. As shown in FIG. 8, supernatants that were diluted to 1:1000 or greater resulted in target populations in which approximately 10% or less of the cells were transduced; in such populations the vast majority of the cells are expected to have single copy proviral integrants. In the transduced populations, the overall fluorescence intensity of the populations were comparable for the hrGFP and EGFP expression vectors. Fluorescence for rGFP was significantly lower than for the latter two genes. Similar results were obtained for experiments involving the transduction of HeLa, CHO, COS7 and NIH3T3 cells (data not shown).

### OTHER EMBODIMENTS

Other embodiments will be evident to those of skill in the art. It should be understood that the foregoing detailed description is provided for clarity only and is merely exemplary. The spirit and scope of the present invention are not limited to the above examples, but are encompassed by the following claims.

1. A humanized polynucleotide, said polynucleotide encoding R. reniformis GFP.

2. The humanized polynucleotide of claim 2, wherein said polynucleotide comprises the sequence of SEQ ID NO: 3.

3. A recombinant vector comprising a polynucleotide of any one of claims 1-3.

4. A cell containing a recombinant vector of claim 4.

5. A method of producing R. reniformis GFP comprising the steps of:
a) introducing a recombinant vector comprising a humanized polynucleotide sequence encoding R. reniformis GFP to a cell;
b) culturing the cell of step (a); and
c) isolating R. reniformis GFP from said cell.

6. The method of claim 6, wherein said humanized sequence encoding R. reniformis has the sequence shown in SEQ ID NO: 3.

7. The method of claim 6 wherein said cell is a bacterial cell.

8. The method of claim 6 wherein said cell is a eukaryotic cell.

9. The method of claim 10 wherein said eukaryotic cell is a mammalian cell.

10. The method of claim 11 wherein said mammalian cells are human.
